(19) United States
(12) Patent Application Publication
(10) Pub. No.: US 2023/0290483 A1
(43) Pub. Date: Sep. 14, 2023

(54) Therapeutics Platform for Mental Health Therapy for People with Disabilities

(71) Applicant: Toivoa Inc., Sarasota, FL (US)

(72) Inventors: Laura Randa, Sarasota, FL (US); Brian Doan, Sarasota, FL (US)

(21) Appl. No.: 18/118,482

(22) Filed: Mar. 7, 2023

Related U.S. Application Data
(60) Provisional application No. 63/444,059, filed on Feb. 8, 2023.
(60) Provisional application No. 63/317,409, filed on Mar. 7, 2022.

(51) Int. Cl. G16H 20/70; G16H 10/60

(52) U.S. Cl. CPC G16H 20/70; G16H 10/60

(57) ABSTRACT

A system for providing disability-tailored therapy to a user having a disability with a presentation of the disability-tailored therapy compliant with accessibility requirements such that the presentation is accessible to the user having the disability is provided. The system includes a memory, a processor coupled to the memory programed with executable instructions, and a user device. The executable instructions cause the processor to obtain a disability-tailored therapy session for the user having the disability based on the disability. The user device configured to receive an input form the user and present the disability-tailored therapy session. The input is user information comprising the disability. The presentation is configured to be accessible to the user having the disability.

### CROSS-REFERENCE TO RELATED APPLICATIONS

This application claims the benefit of U.S. Provisional Application Ser. No. 63/317,409 titled Digital Therapeutics Platform for Mental Health Therapy for Disabilities filed Mar. 7, 2022 and U.S. Provisional Application Ser. No. 63/444,059 titled Therapeutics Platform for Mental Health Therapy for Patients with Disabilities filed Feb. 8, 2023, each of which is hereby incorporated by reference herein in its entirety.

### TECHNICAL FIELD

The present disclosure relates generally to a platform for providing therapy and, more specifically, a therapeutics platform for mental health therapy for people with disabilities.

### BACKGROUND

Behavioral health in America is in a state of crisis. Individuals meeting the criteria for a mood disorder has never been higher at 40% of the US population. Adults with disabilities report experiencing frequent mental distress almost 5 times as often as adults without disabilities. The development of digital mental health intervention will enable widespread access to critical therapeutic regimens and help overcome both stigmas and practical barriers that hold many people back from seeking treatment.

Today almost 26% of adults in the U.S. live with some sort of disability with 23 million reported to have behavioral health issues. Access to quality care is hindered by long waitlists and underserved communities. Digitally based therapeutics have seen an upswing in use to address this gap. However, these platforms are often lacking the accessibility elements a user with a disability typically requires such as larger text, magnification, screen-reading custom vibrations, flash alerts, mono-audio, color, closed captioning, keyboard accessibility, alternative text, voice assistant, text to speech/voice recognition, and contrast. According to Ex-Congressman Tony Coehlho, “98% of the million most common webpages are inaccessible in some fashion.” Aquino, Steven, “Ex-Congressman Tony Coelho Opens Up About Being Disabled And Where The Americans With Disabilities Act Fails In A Digital, Pandemic-Ruled Age,” Forbes.com, 22 May 2020, https://www.forbes.com/sites/stevenaquino/2020/05/22/ex-congressman-tony-coelho-opens-up-about-being-disabled-and-where-the-americans-with-disabilities-act-fails-in-a-digital-pandemic-ruled-age/. As a result, these digital health platforms have not seen effective use across patients with disabilities.

Research has shown the breadth of barriers that people with disabilities face. Several studies find persistent barriers in telehealth accessibility. A study of 74 Deaf participants who had recently used telehealth found that 65% of participants experienced communications accessibility barriers. Deaf patients frequently experience the inability to connect remote medical interpreters or real-time captioners through the secure telehealth platform, the inability to see the provider on video, and other technical issues, including poor audio quality. Another study found that of 285 blind and low vision participants who had used telehealth to meet with their healthcare provider, 21% reported the telehealth platform was not accessible with their assistive technology, and preliminary data from a forthcoming study suggests that the number could exceed 50% a year later. Moreover, while there are no studies directly examining the telehealth experiences of DeafBlind people, anecdotal reports suggest that the vast majority of DeafBlind people are completely unable to utilize telehealth as it currently exists.

The challenges are present in every sector of society. Nearly 60% of the educators surveyed in a Fall 2020 study reported their blind and low vision students could not access one or more of the digital learning tools they were expected to use in class. A 2022 study found that about 50% of survey respondents experienced accessibility challenges when filling out electronic onboarding paperwork. Moreover, an annual automated analysis demonstrates how common inaccessibility barriers are, finding that of one million webpages reviewed in 2021, 97% had accessibility issues, and an average of 50 errors appeared on every page.

These findings are neither exhaustive of all website-related issues nor comprehensive of the entire disability community. The disability community is large and diverse, facing access issues that continue to grow and evolve with the ever-changing landscape of websites and applications. While the studies cited primarily explored the experiences of people with sensory disabilities, accessibility issues are pervasive, frequent, and harmful for people with other disabilities as well.

The scale of inaccessibility and its impact on access to nearly every type of web or application-based activity necessitates regulatory action. As the COVID-19 pandemic has made abundantly clear, we live in a society that increasingly lives and works through digital tools and online spaces. When websites and applications are inaccessible, people with disabilities cannot apply for jobs, work efficiently, attend school, access healthcare, schedule a ride, shop, find public health information, apply for public benefits, and more. Due to the enormous challenge for people with disabilities, a U.S. senator and a U.S. congressman have even introduced tech equity legislation. “Duckworth, Sarbanes Introduce Bicameral Legislation to Help Make Websites and Software Applications Accessible for Americans with Disabilities”, Duckworth.Senate.gov, 29 Sep. 2022, https://www.duckworth.senate.gov/news/press-releases/duckworth-sarbanes-introduce-bicameral-legislation-to-help-make-websites-and-software-applications-accessible-for-americans-with-disabilities. As the COVID-19 Pandemic caused a surge in anxiety and depression, digital mental health apps and the use of telemedicine were utilized in lieu of in-person visits.

People with disabilities have persistently been excluded from digital spaces covered by the ADA. At least five barriers exist with patients with one or more disabilities in the current technologies of the prior art: attitudinal uniqueness, organizational or systemic intuitiveness, architectural or physical platform, information or communications style, and technology with accommodations.

### SUMMARY

Embodiments of the present invention address and overcome one or more of the above shortcomings and drawbacks, by providing methods, systems, and apparatuses related to a therapeutics platform for mental health therapy for disabilities. Additional features and advantages of the invention will be made apparent from the following detailed description of illustrative embodiments that proceeds with reference to the accompanying drawings.

In an exemplary embodiment, a system for providing disability-tailored therapy to a user having a disability with a presentation of the disability-tailored therapy compliant with accessibility requirements such that the presentation is accessible to the user having the disability includes a memory; a processor coupled to the memory programed with executable instructions, the executable instructions cause the processor to obtain a disability-tailored therapy session for the user having the disability based on the disability; and a user device configured to receive an input from the user and present the disability-tailored therapy session, wherein the input is user information comprising the disability, and wherein the presentation is configured to be accessible to the user having the disability.

In another exemplary embodiment, a computer-implemented method for providing disability-tailored therapy to a user having a disability with a presentation of the disability-tailored therapy that is compliant with accessibility requirements such that the presentation is accessible to the user having the disability in a data processing system comprising a processor and a memory comprising instructions that are executable by the processor includes receiving, by the processor, user information, wherein the user information comprises the disability of the user; selecting, by the processor from a database, a disability-tailored therapy session based on the disability; and presenting, by the processor, the disability-tailored therapy session, wherein the presentation is compliant with the accessibility requirements such that the disability-tailored therapy session is accessible to the user having the disability.

In yet another exemplary embodiment, a non-transitory computer-readable medium having stored thereon instructions for providing disability-tailored therapy to a user having a disability with a presentation of the disability-tailored therapy that is compliant with accessibility requirements such that the presentation is accessible to a person having the disability, which when executed by at least one processor, cause the processor to receive user information, wherein the user information comprises the disability of the user; select, from a database, a first disability-tailored therapy session based on the disability; and present the first disability-tailored therapy session, wherein the presentation is compliant with the accessibility requirements such that the first disability-tailored therapy session is accessible to the user having the disability.

Additional features and advantages of the invention will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the invention, there are shown in the drawings embodiments that are presently preferred, it being understood, however, that the invention is not limited to the specific instrumentalities disclosed. Included in the drawings are the following Figures:

FIG. 1 is a flowchart of a method of providing therapy to a user having a disability, according to an embodiment of the disclosure;

FIGS. 2A and 2B illustrate aa user inputting his or her disability into the platform, according to an embodiment of the disclosure;

FIGS. 3A-3H each illustrate an embodiment of modified therapy sessions for people with hearing loss and a mobility disability, according to an embodiment of the disclosure;

FIGS. 4A-4HH show exemplary embodiments of screenshots of the platform's WCAG-compliant presentation, according to an embodiment of the disclosure;

FIGS. 5A-5R show exemplary embodiments of screenshots of the platform's WCAG-compliant user interface, according to an embodiment of the disclosure;

FIG. 6 is a flowchart of a method of matching a user with a coach, according to an embodiment of the disclosure;

FIGS. 7A-7L illustrate how a user and a coach may communicate, according to an embodiment of the disclosure;

FIG. 8 illustrates a use-case diagram of how the platform works, in an embodiment of the disclosure;

FIGS. 9A-9C illustrates the platform's master flow diagram of the platform, according to an embodiment of the disclosure;

FIGS. 10A-10D illustrate the platform's information architecture (i.e., flow), according to an embodiment of the disclosure;

FIG. 11 illustrates a sub-routine diagram, according to an embodiment of the disclosure;

FIGS. 12A-12C illustrate the platform's architecture diagram, according to an embodiment of the disclosure;

FIG. 13 illustrates the platforms tech flow, according to an embodiment of the disclosure;

FIG. 14 illustrates the platform's cloud infrastructure, according to an embodiment of the disclosure;

FIG. 15 is the platform's VPC diagram, according to an embodiment of the disclosure; and

FIG. 16 illustrates an exemplary computing environment, according to an embodiment of the disclosure.

This summary is provided to introduce a selection of concepts in a simplified form that are further described below in the detailed description. This summary is not intended to identify key features or essential features of the claimed subject matter, nor is it intended to be used to limit the scope of the claimed subject matter. Additional features and advantages of the disclosed technology will be made apparent from the following detailed description of illustrative embodiments that proceeds with reference to the accompanying drawings.

### DETAILED DESCRIPTION

The present disclosure discloses a therapeutics platform for mental health therapy for people with disabilities. The platform administers therapy in a digital format with augmentation through live coaches and peer-to-peer social networking. In some embodiments, the therapy is provided in a modular personalized incremental dose format which can be consistent with typical use patterns for mobile devices. The user experience (UX) is by design consistent with Web Content Accessibility Guidelines (WCAG 2.1 and later versions, including e.g., WCAG 2.2 and WCAG 2.3, for example and not limitation) to ensure seamless use by persons with disabilities. Both therapy and WCAG features are tuned to the user's self-reported disability. Progress is tracked using a series of validated psychological assessments and recorded in a HIPPA-compliant server. In some embodiments, the platform is embodied by a system, a computer-implemented method, and a non-transitory computer-readable medium. This platform allows for quick and remote therapy. It makes mental health treatment available, affordable and accessible for populations of patients with one or more disabilities when and where they choose. It solves the technical problem of timely access to effective anxiety and/or depression therapy for persons with disabilities by providing a design system that can administer remote digital therapy in a manner accessible to someone with disabilities.

The number of qualified individuals to perform therapy is far below the number of people seeking treatment. This can be true because, for example, qualified individuals are not accepting new patients or because there are few or no qualified individuals in a patient's area (e.g., in a rural area). To illustrate how big of a problem this is, consider the following: 65% of psychologists have no capacity for new patients, “2021 COVID-19 Practitioner Survey: Worsening Mental Health Crisis Pressures Psychologist Workforce,” www.apa.org, 19 Oct. 2021, https://www.apa.org/pubs/reports/practitioner/covid-19-2021, and 60% of rural Americans live in a designated mental health provider shortage area. “Mental Health and Rural America: Challenges and Opportunities,” www.nimh.nih.gov, 30 May 2018, https://www.nimh.nih.gov/news/media/2018/mental-health-and-rural-America-challenges-and-opportunities. This results in a huge challenge to patient care. A solution may involve providing a computer-implemented method for providing therapy because it does not require a qualified individual. If a solution does not require a qualified individual, of which there are not enough, more people may have access to therapy. In addition, a user may use a computer-implemented method more than he or she would attend therapy with a qualified individual because the user can more easily fit the computer-implemented method into his or her schedule because the computer-implemented method can be practiced from anywhere and for any amount of time.

However, providing a computer-implemented method comes with several obstacles. First, computer-implemented therapy may be generic to the general population and therefore not as effective for persons with disabilities. Second, a computer-implemented method may not be accessible to persons with disabilities. Third, a computer-implemented method may lack a personal touch that can be helpful to encourage users in their treatment.

This application discloses a therapeutics platform for mental health therapy for people with disabilities. The platform can provide therapy tailored to a person having a particular disability for treatment of a particular condition, disease, or diagnosis, be accessible to people with disabilities, match users with coaches to encourage the users, and provide a community component where users can connect to discuss their treatment.

One aspect of the platform's uniqueness is borne out of the focus on accessibility: Other websites and apps are designed for general use with accessibility tacked on at the last moment for ADA compliance/legal purposes. The Program is designed for use by persons with disabilities by first intent and all user experience is WCAG compliant by default. Another aspect of the platform's uniqueness is borne out of the focus on persons with disabilities: Therapy modules are designed to resonate with our target community and involve scenario evaluation that are specific to one's disability. This platform is the first SaMD approved for anxiety and depression with a focus on persons with Disabilities. A large+10M population with minimalized access to therapy for anxiety and depression.

THERAPY—The platform can provide therapy tailored to a person having a particular disability for treatment of an ailment. This platform is not limited by the type of therapy. In some embodiments, the therapy can be intended to modify thinking patterns that trigger anxiety and depression for people with disabilities. In some embodiments, the therapy can be one or more of applied behavior analysis, client-directed outcome informed therapy, cognitive behavioral therapy, mindfulness-based cognitive therapy, and solution-focused brief therapy. The platform is not limited by the type of ailment the therapy seeks to treat. In some embodiments, the therapy can treat one or more of a user's anxiety and depression. The platform is not limited by the type of disability for which the therapy is tailored. In some embodiments, the disability is an impairment related to vision, hearing, mobility, cognition, self-care, independent living, and long-COVID. In some embodiments, the platform is tailored for adolescent indication (12 to 21) and/or adult indication (22 to 64).

Embodiments deliver therapy digitally tuned for the disability community and subgroups within. This therapy includes, but not limited to, Cognitive Behavioral Therapy at times and in settings most comfortable and convenient for the individual seeking treatment for anxiety. The systems and methods described herein reduce negative interpretations and reduced the symptoms of anxiety. Embodiments provide adaptations to improve the ease of use, functionality, and visual appeal of the app. Further a “personalized incremental dose” model is advanced that offers more frequent, brief (˜5 minute) focused doses of training that can be integrated into the user's daily life, and which better fits how people use apps on their phones. Finally, the application adds capabilities that offer personalized, targeted care in multiple domains, to increase both adherence and efficiency of care to multiple populations. Embodiments help reduce the severe mental health burden of anxiety and depressive disorders for patients with one or more disabilities, by providing access to care to a broad portion of the population, cost effectively, and at the times and locations that are most useful to end-users. Embodiments combine therapy, technology, disability processes, people expertise to make digital therapy accessible—and reduce anxiety and depression. The training and ongoing compliance/monitoring combined with the platform will help the group maintain long term success to build with accessibility in mind.

In some embodiments, the therapy is delivery in modules. In some embodiments, there are several, e.g., eight, modules, including an introduction video, situational training, and retention quizzes. The modules can be designed so sub-sections can be completed across multiple sessions, in some embodiments ˜15 minute sessions, where the user can complete as many modules at a time as they see fit. Each of the modules can be paused and resumed at any time, which can give the user the flexibility to work through the therapy at his or her own pace. Each module can contain one or more descriptions of a situation that is likely to elicit an emotional response from the user; one or more questions, and in some cases, prompts to submit a response, to help the user identify, for example, the user's common cognitive distortions; questions to confirm the user's understanding of the module's teachings; suggestions for activities the user can perform to practice prior to the next module; and questions to prompt the user to identify the challenges that prevented the user from completing the suggested activities and solutions the user can use to overcome the challenges. Further, in some embodiments, one or more modules can have one or more purpose. The purpose of a module can be, for example, to teach the user a skill or challenge in the user's treatment for the ailment for which the user is receiving treatment. For example, a purpose of a module may be to teach the user about behavioral awareness, as will be described in further detail below.

In an embodiment, the platform provides Cognitive Behavioral Therapy (CBT) to treat a user's anxiety over several modules. CBT is a proven therapy for improvement in Anxiety and Depression in clinical settings. Over the past decade, both websites and mobile app software been demonstrated to be an effective tool for delivery of CBT. Part of CBT relies on the patient to envision different scenarios and practice mechanisms for situational responses. For the patient with disabilities, the hypothetical scenarios are not personally relatable so the emotional responses required for CBT are not “triggered”. In the therapy provided by the platform, these hypothetical situations have been created with the help of psychologists who specialize in treatment of persons with disabilities. The hypothetical situations are also tailored to a person's self-identified disability and include examples such as “imagine you are needing to enter a building and there is no wheelchair ramp” (mobility) or “imagine you are having a conversation where the use of masks makes it difficult to read lips” (hearing). These disability-tuned scenarios will help trigger the emotional responses that the therapy is looking to acknowledge.

This therapy reduces negative interpretations and reduces the symptoms of anxiety. Valuable learnings are incorporated into the development of our platform, building on work established treatment content, infrastructure, delivery platform, and history of efficacy allows for considerable efficiency in advancing the research and development needed to bring the program to readiness for commercialization. By targeting the tendency to assign negative, threatening meanings to ambiguous situations, we engage a target that is a transdiagnostic mechanism underlying multiple emotional disorders. This promises to be a highly efficient approach to address not only anxiety symptoms (the primary symptom outcome) but also highly comorbid symptoms domains, such as depression, which are also maintained by mechanisms involving negative, repetitive thinking.

In one module, the therapy can include teaching the user that thoughts can create or intensify negative emotions and end up harming the user's mental health, about behavioral avoidance, which happens when short-term comfort takes precedence over long-term growth, and why goal setting is so important and how creating attainable, measurable, and personal goals can help the user on his or her journey to a more productive, happy, and fulfilling life. Further, the therapy can teach the user about behavioral activation, and why it's important to figure out ways to engage in more pleasant and gratifying activities throughout the day. The therapy can also introduce the user to an activity log, which can be a tool for increasing the user's behavioral awareness, which can help the user figure out which activities the user participates in that are associated with the greatest improvement in the user's mood. The platform can also prompt the user to complete an activity log tracking the user's activities and examine that activity log for areas in which the user's moods and activities are connected and suggest alternate behaviors to begin to change in order to improve the user's moods, and ultimately, decrease the user's anxiety and/or depression. In some embodiments, a user device, e.g., a cell phone, a tablet, a computer, may periodically send an alert to the user to remind the user to enter information into the activity log. The therapy can also prompt the user to schedule one or more activities that the user would find pleasant or offer the user a sense of accomplishment. Further, the therapy can discuss sleep and how it can affect those struggling with depression and anxiety. It can also teach the user steps the user can take to improve the user's sleep, e.g., a regular bedtime and wake time, plan wind-down time before bed, and brainstorm ideas about how to handle times when it's hard to fall asleep or the user wakes up in the middle of the night.

In another module, the therapy can teach the user behavioral strategies to help the user feel more empowered as the user starts scheduling more helpful activities throughout the week. One behavioral strategy can be to try to be open and curious about what might be interesting to the user. The therapy may suggest to the user to try a new behavior a few times, e.g., three times, because it can take repetition to figure out what really works for the user. Another behavioral strategy is to break down a task into small parts, which can be a good way to make a task more manageable and to help the user overcome what is often such a critical barrier: getting started. Yet another behavioral strategy is to make a commitment to the activities the user schedules by writing them down, setting reminders, asking loved ones to keep you accountable, and always thinking ahead about what might get in the way. Yet another behavioral strategy is to follow through on activities that the user plans regardless of how the user feels in the moment.

In another module, or in one of the modules discussed above, the therapy can teach the user about the role of avoidance. Understanding how avoidance operates can be key to breaking avoidance patterns. People can use avoidance to manage short term challenges, but that tends to cause longer-term problems. Avoidance can interfere with accomplishing our long-term goals and, in turn, perpetuate negative emotions. The therapy can pay special attention to rumination as avoidance and how that can keep people locked in a very negative thinking pattern. The therapy can teach the user a way to break patterns of avoidance by recognizing the trigger, response, and avoidance pattern, so the user can experiment with replacing avoidance patterns with healthier coping strategies that will serve the user's long-term goals.

In another module, the therapy can teach the user about thought records, and how they can help the user reassess emotionally charged situations. The therapy can prompt the user to choose a situation, identify his or her emotions, and figure out his or her automatic thoughts. It can be important to use the thought record throughout the week. Writing things down can be powerful when trying to make changes, because it can help a user process things more fully and can allow the user to work through the thought record systematically.

The therapy can also teach the user how to come up with alternative responses on the thought record. It can be important to recognize that one's thoughts may or may not be true, and to keep an open mind when evaluating thoughts. The therapy can teach the user the process of using past evidence and thoughts about a friend in a similar situation to create alternative responses. The therapy can also prompt the user to the thought record every day for a period of time, e.g., a week, because practice can be a good way to get better at recognizing automatic thoughts and alternate responses.

As mentioned above, each module can contain one or more descriptions of a situation that is likely to elicit an emotional response from the user. The situations described can be disability-specific in that it describes a person with a disability in a situation that would be stressful to the person because of his/her disability. Because the user and the person described in the situation have the same disability, the user can empathize with the stressor described in the situation. For example, the therapy session may describe the following situation and use it to teach the user how scheduling pleasant and/or rewarding activities can help the user with his or her anxiety or depression:

“Tom has been meaning to make friends in the new city he moved to 3 months earlier. However, he always feels hesitant to meet new people, because it can be hard to hear them in crowded places due to his hearing loss after working for 25 years in the music industry. Instead of abandoning the idea that he'll never meet new friends, we would suggest to him to choose two music stores in the area to visit that week to learn more about the music scene and engage with people with similar interests. Although they may be playing music in the store, it'll likely be a lot less noisy than a concert or show—the types of places he used to meet him friends. And by engaging with locals in the area, he may meet people with whom he has mutual interests. And who knows—they may even have similar hearing issues if they worked in the business, which could also help him feel less alone. After he visits two shops, he'll likely feel both a sense of accomplishment and less anxiety about ways to meet new people. Plus, even if he meets just one cool person, who knows who else he might meet through that person, which might give him the confidence to meet more people the following week.”

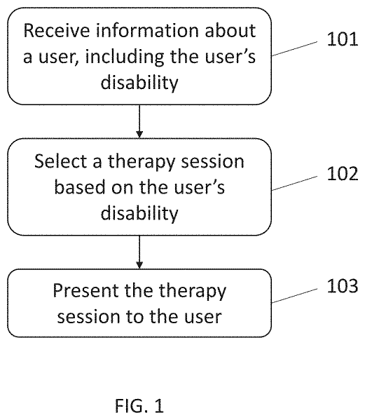

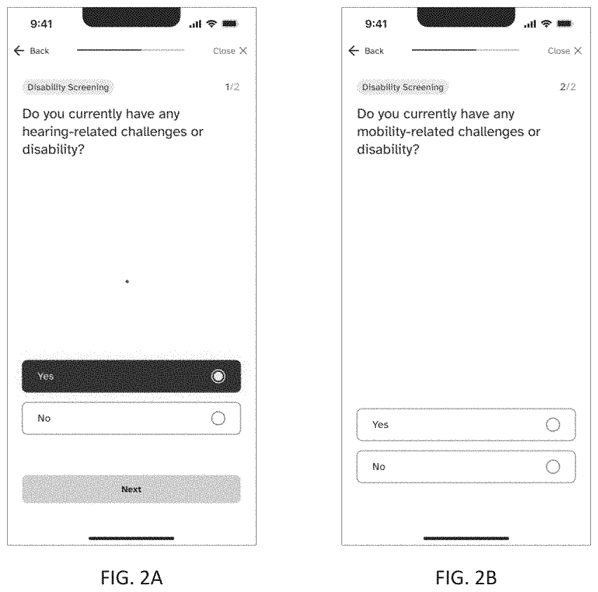

FIG. 1 is a flowchart of a method of providing therapy to a user having a disability, according to an embodiment of the disclosure. In some embodiments, the platform can perform a method of providing therapy to a user having a disability. A user can input his or her information into the platform. The information can include, for example, the user's disability, age, gender, sex, race, learning style, and socioeconomic information. An example of a user inputting his or her disability is illustrated in FIGS. 2A and 2B, according to an embodiment of the disclosure. At step 101, the method can include this receiving information. At step 102, the method can include selecting a therapy session based on the user's disability. At step 103, the method can include presenting the therapy session to the user.

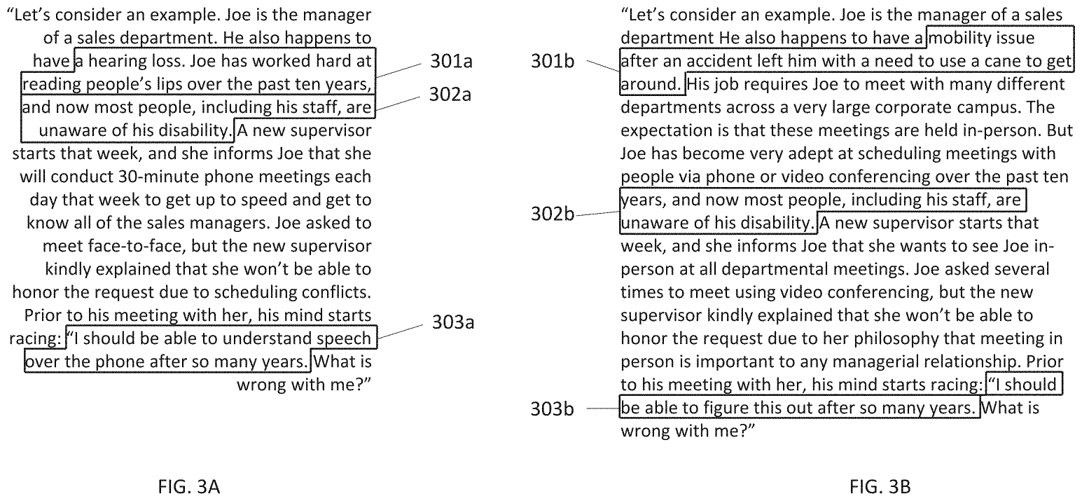

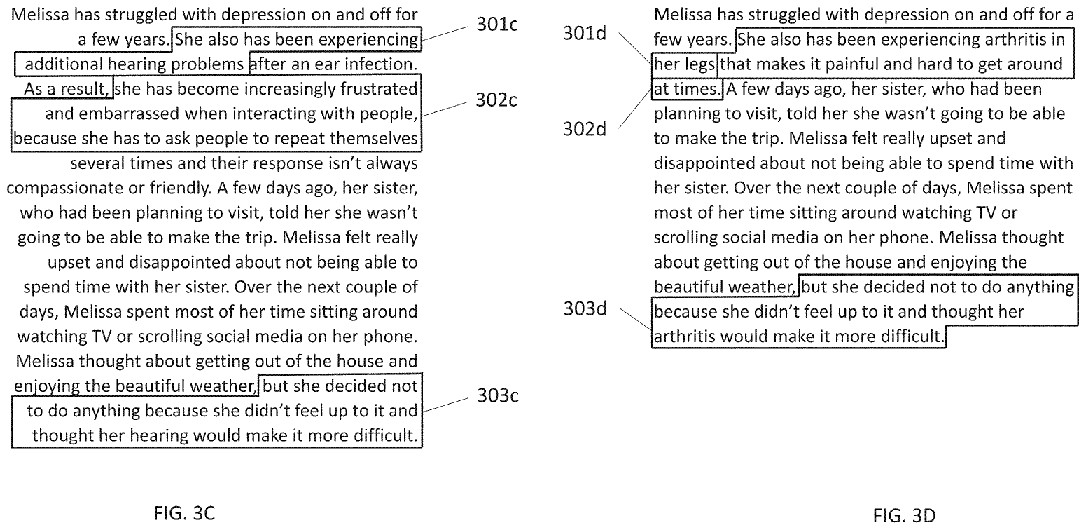

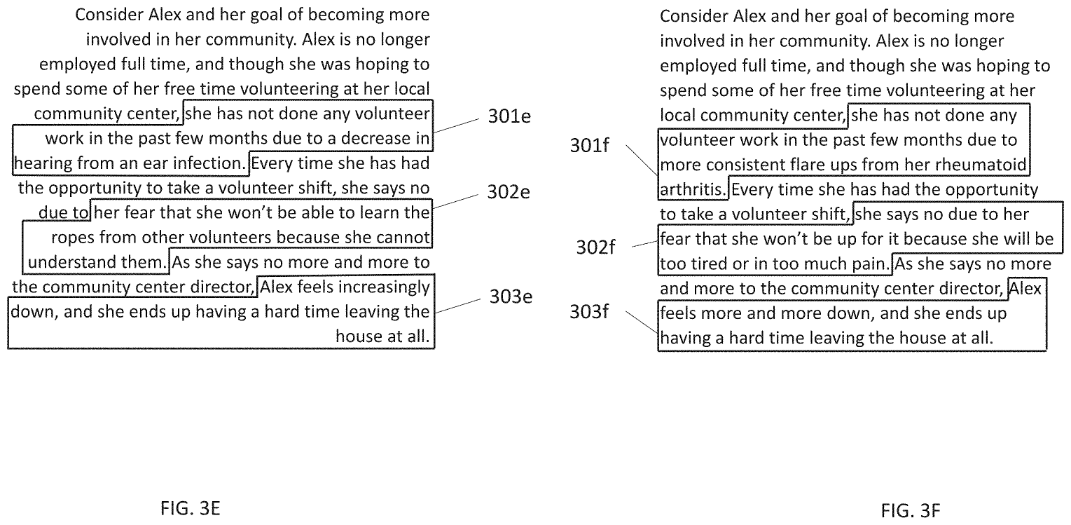

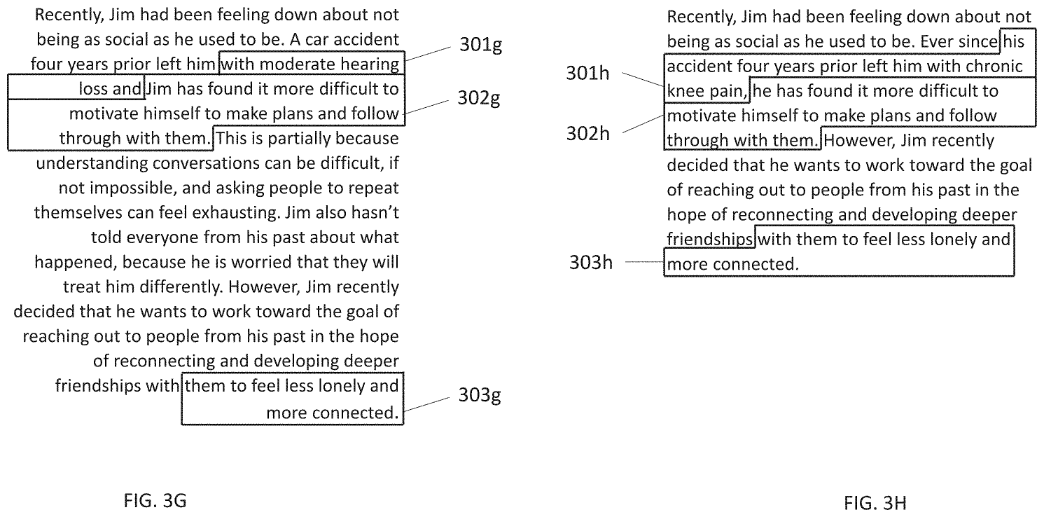

In some embodiments, selecting a therapy session based on a user's disability can include one or more of the following steps. The method can include retrieving a therapy session from a database. The therapy session may include one or more of the following sections: a stock language section, an adjusted section, a relatable section, and a stressor section. The method can further include retrieving a disability-specific insert and modifying the therapy session by inserting the disability-specific insert into the adjusted section of the therapy session. FIGS. 3A-3H each illustrate an embodiment of modified therapy sessions for people with hearing loss and a mobility disability, according to an embodiment of the disclosure.

In some embodiments, the mobile application platform utilizes a personalized incremental dose model because completion of one hour sessions on a user device like a cell phone can difficult and can result in low adherence and retention not 15 to 20 minutes. A “personalized incremental dose” model offers more frequent, brief (˜5 minute) focused doses of training. The personalized incremental dose model is important because requiring completion of 15-20 minute-long modules is associated with low adherence and high attrition. Switching to a personalized incremental dose approach is also an adaptation that aligns with the way people naturally use their mobile devices. No other applications offer personalized, sequenced personalized incremental doses to target anxious thinking and associated anxiety symptoms.

Finally, the application supports a maximally uniform experience across Android and iOS platforms, and allows for easy modification and personalization for a suite of digital interventions, laying the groundwork for a “digital apothecary” that can meet the needs of very diverse subpopulations of patients with one or more disabilities.

Creation and validation of a “personalized incremental dose” delivery format better matches the ways people use mobile phones (as compared to a therapy-session style delivery model).

Development of personalized interventions, including timing for the personalized incremental dose interventions to optimally fit into a person's day and provide care when and where it is most needed, and in terms of the stressor domain targeted by each personalized incremental dose to ensure it is personally relevant and meet's the user's most pressing need.

ACCESSIBILITY—To ensure that the users to whom the therapy sessions are tailored are able to go through the core user flows and patient journey seamlessly, the therapy sessions can be accessible to users. To illustrate, consider a user who has anxiety and a vision impairment. The therapy session can describe a situation that would be stressful to a person having a vision impairment, and the therapy should also be accessible to the user having the vision impairment. For example, the therapy session can be presented to the user audibly. In some embodiments, this platform can be accessed by all patients with a disability. To illustrate the impact this has, consider that there are 61 million patients with a disability 19% of Americans.

In most all application software, accessibility features are added late in development with an intent to mitigate ADA policy legal compliance issues, rather than as a product requirement. In the platform, design elements have been incorporated into our system requirements at the onset to ensure conformation to the universal design principles and accessibility standards like the Web Content Accessibility Guidelines Version 2.1 (WCAG 2.1) and previous and later accessibility standards, including, for example and not limitation, WCAG 2.2 and WCAG 2.3. The WCAG 2.1 guidelines were developed to ensure that a digital platform is Perceivable (can you observe it), Operable (can you use it), Understandable (Can it engage with the user in a predictable manner) and Robust (is it interpretable by assistive technologies). Even with these standards, a 2022 study showed 96.8% of global websites had WCAG 2.1 access deficiencies. By first intent, the platform's design elements were tested against standards during development and include features such as no flashing lights, a 3:1 contrast ratio for colors, and closed-captions for all audio/video media. In some embodiments, the platform undergoes a rigorous assessment by and is certified as compliant by Trusted Testers certified by the Department of Homeland Security's Office of Accessible Systems & Technology. This certification guaranteed full compliance to accessibility standards and legal requirements.

In some embodiments, the platform is compliant with accessibility requirements. Accessibility requirements may include one or more of the following: mobile accessibility standards, perceivable information and user interface, operable user interface and navigation, understandable information and user interface, robust content and reliable interpretation, and content is compatible with current and future user tools. Perceivable information and user interface may include text alternatives for non-text content, captions and other alternatives for multimedia, content can be presented in different ways, and content is easier to see and hear. Operable user interface and navigation may include functionality is available from a keyboard, users have enough time to read and use the content, content does not cause seizures and physical reactions, users can easily navigate, find content, and determine where they are, and users can use different input modalities beyond keyboard. Understandable information and user interface may include text is readable and understandable, content appears and operates in predictable ways, and users are helped to avoid and correct mistakes.

In some embodiments, the delivery of the therapy adheres to one of, some of, or all of the published web content accessibility guidelines (“WCAG”). For example, in some embodiments, the delivery of the therapy described herein has the following features: all audio and video includes text subtitles; all pre-recorded audio and video offer variable playback speeds, ability to stop pause or hide and control volumes independent of system volumes; audio descriptions are available for infographics; material is presented in a linear fashion with text-based function keys, including action prompts; operation can occur in landscape or portrait modes; the purpose of input field is clearly defined; the visual presentation of text and images of text has a contrast ratio of at least 4.5:1; text can be resized up to 200% and images of text shall not be utilized in design; user interface components and graphical objects shall have a color-contrast ratio of at least 3:1; line height (line spacing) is at least 1.5 times the font size; spacing following paragraphs to at least 2 times the font size; letter spacing (tracking) to at least 0.12 times the font size; word spacing to at least 0.16 times the font size; hidden content shall incorporate a “dismiss” function rather than disappear with scrolling; no “timeouts” for entry purposes; flashing icons/media are not utilized; titles, headings and labels shall describe topic or purpose; screen to screen sequence flow (rather than menus) are utilized for logic flow; user interface components with labels that include text or images of text shall contain the text that is presented visually; the human languages for phrases, passages or page can be selected by the user; changes of settings or focusing in a certain area do not change the context of the page; components that have the same functionality within a set of web pages are identified consistently and in similar order; labels or instructions are provided when content requires user input; when an input error is automatically detected, the item that is in error is identified and is described to the user in text (where applicable for security protection) along with corrective suggestions; for security information (name, email, access code), a mechanism is available for reviewing, confirming, and correcting information before finalizing the submission; and in content implemented using markup languages, status messages can be programmatically determined through role or properties such that they can be presented to the user by assistive technologies without receiving focus.

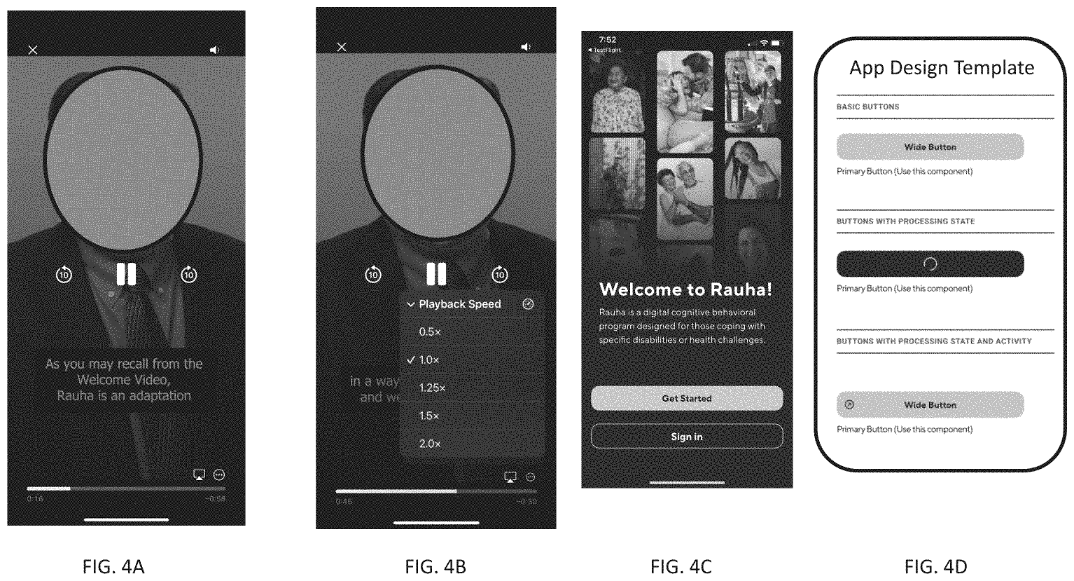

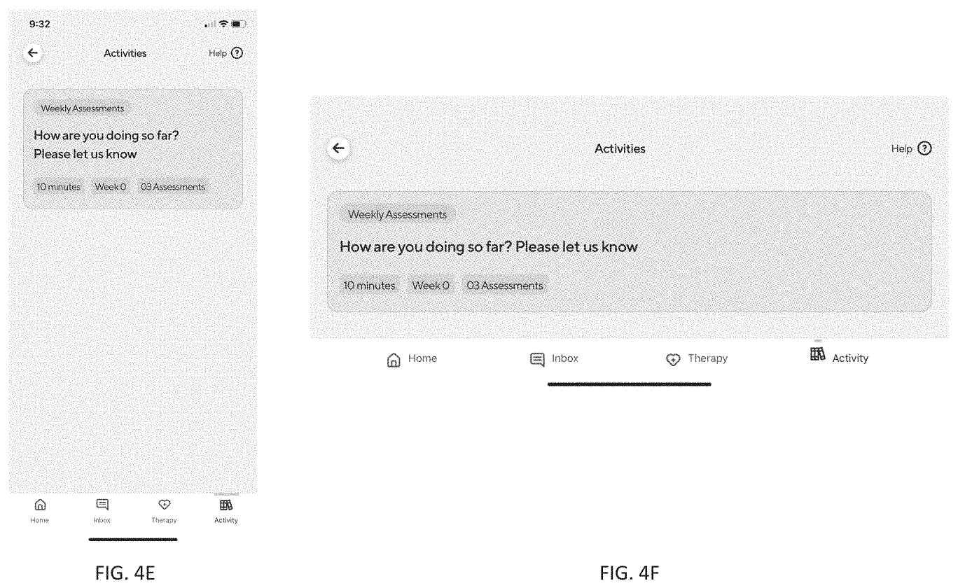

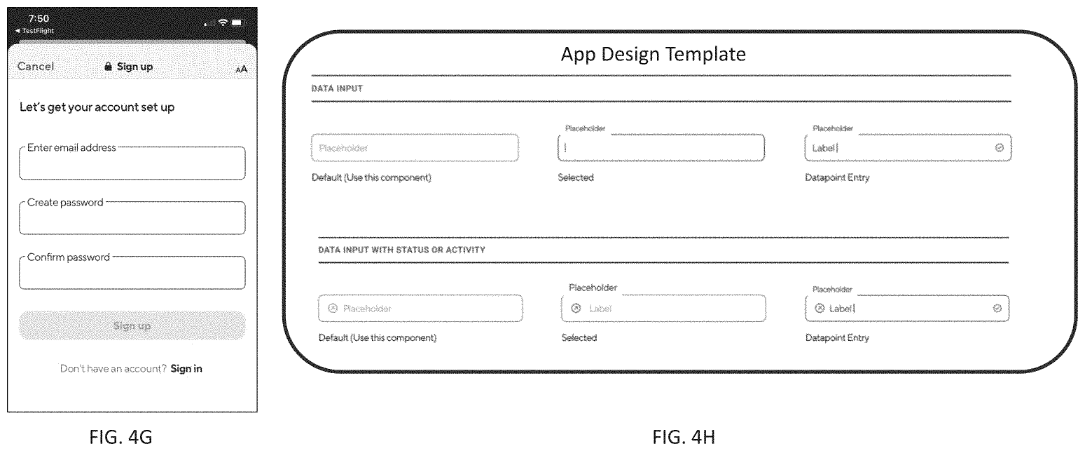

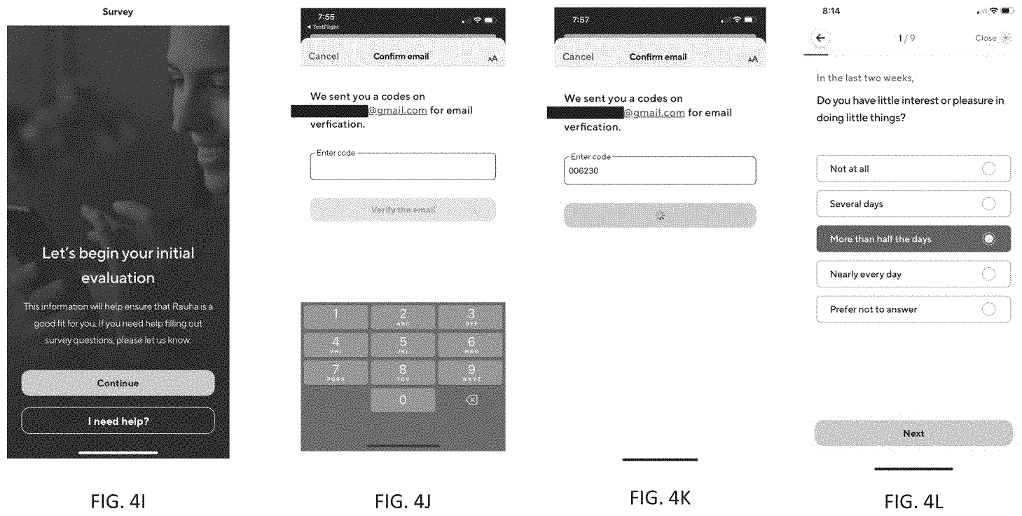

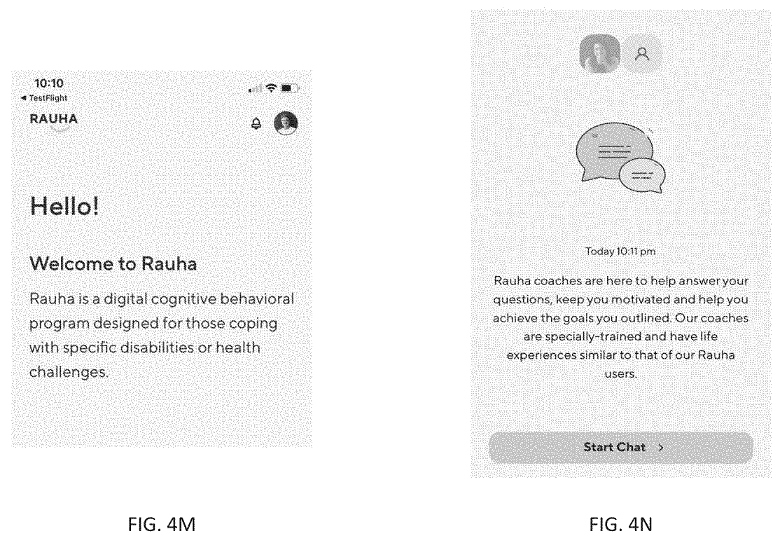

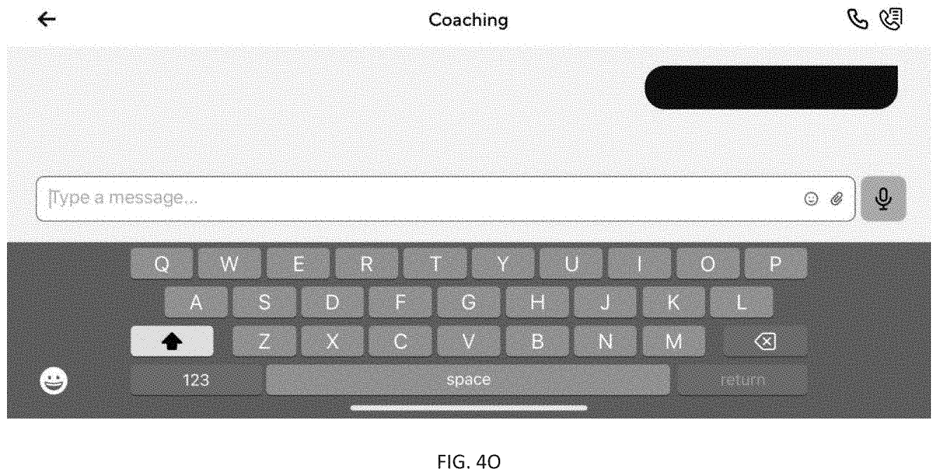

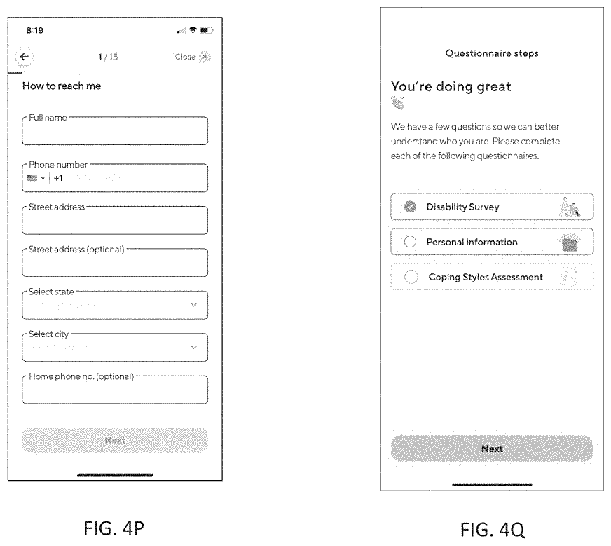

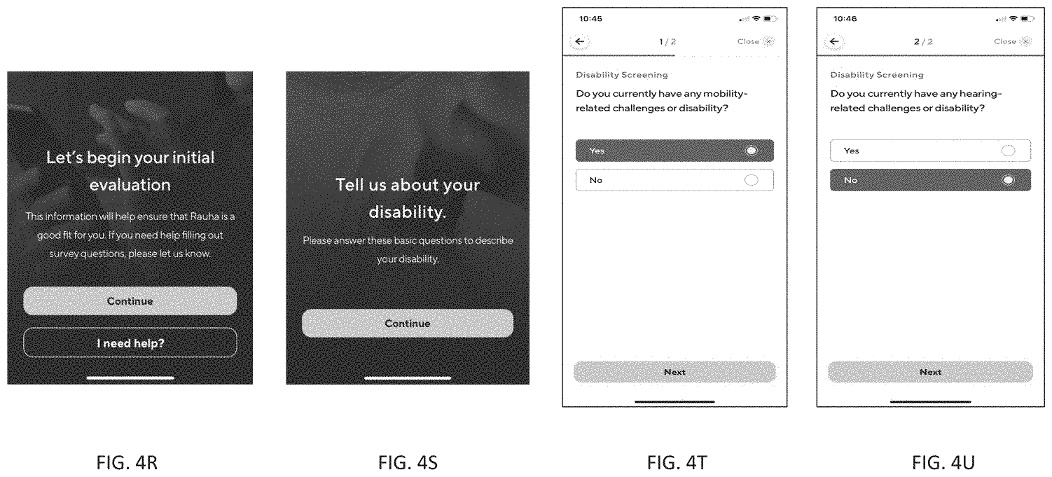

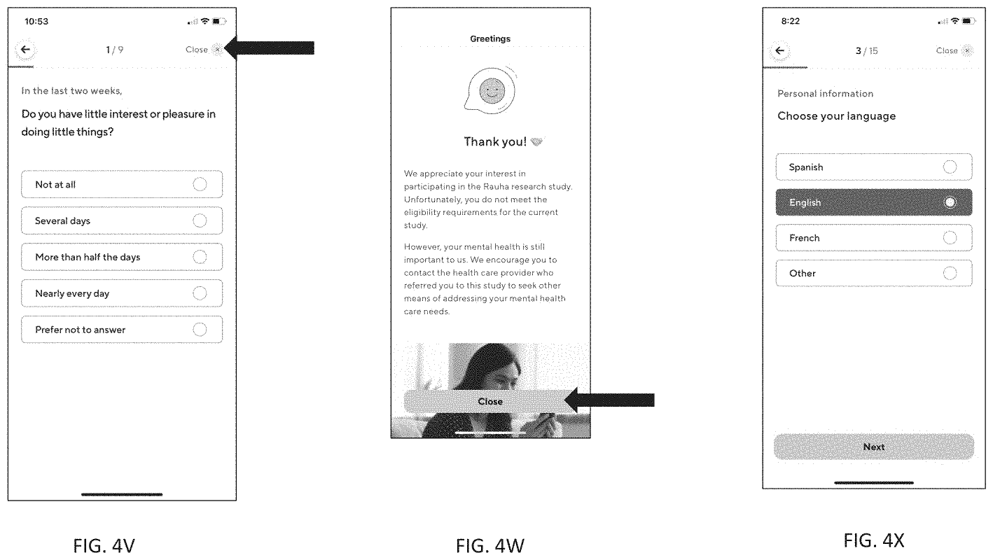

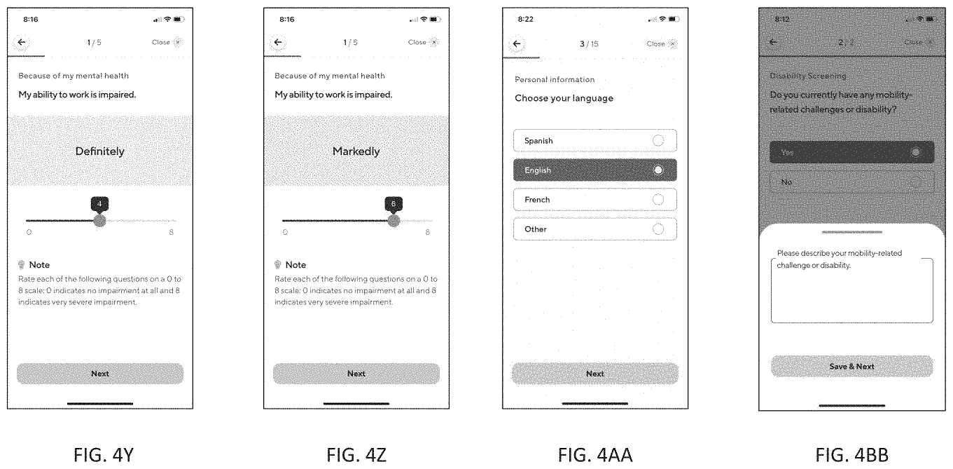

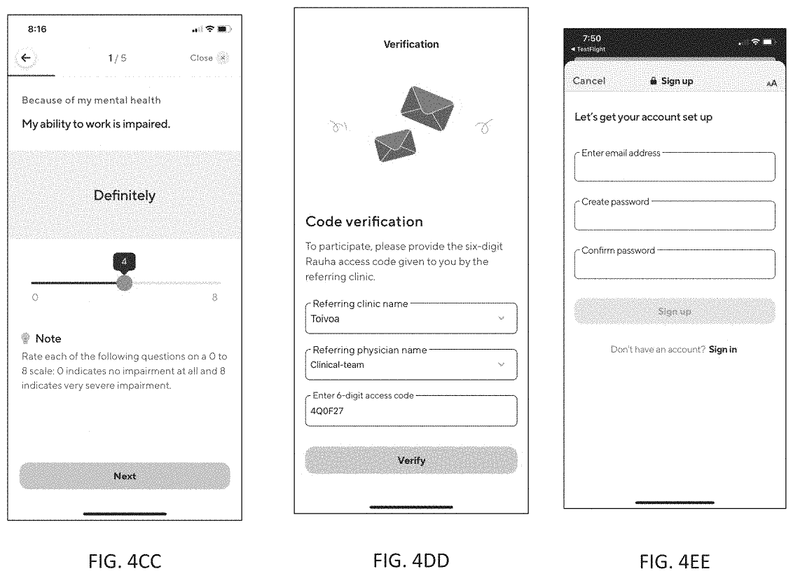

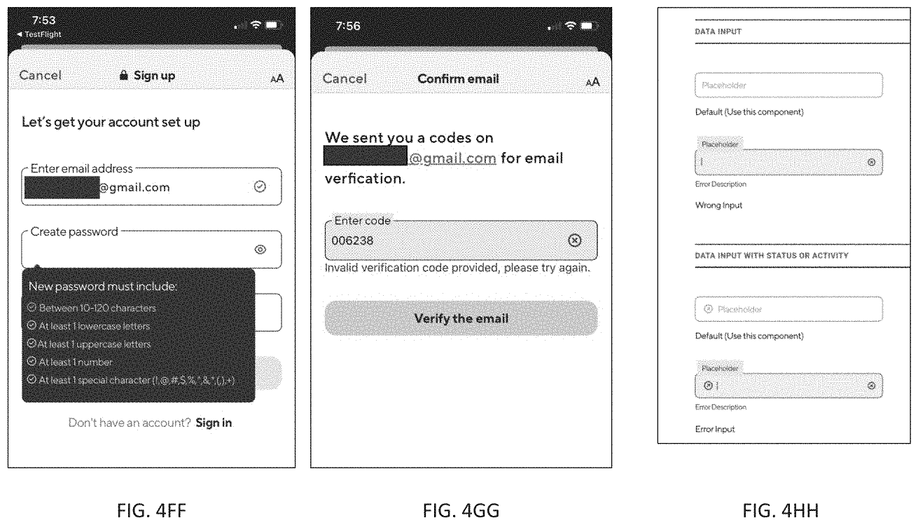

FIGS. 4A-4HH show exemplary embodiments of screenshots of the platform's WCAG-compliant presentation, according to an embodiment of the disclosure. For example, FIG. 4A, illustrates an embodiment in which all audio and video include text subtitles, according to an embodiment of the disclosure. As shown in FIG. 4A, all non-text content that is presented to the user has a text alternative that serves the equivalent purpose, except for input or controls, tests, sensory, CAPTCHA or decoration (WCAG 1.1.1), and that captions are provided for all prerecorded audio content in synchronized media, except when the media is a media alternative for text and is clearly labeled as such (WCAG 1.2.2).

FIG. 4B illustrates an embodiment in which all pre-recorded audio and video offer variable playback speeds, ability to stop pause or hide and control volumes independent of system volumes. As shown in FIG. 4B, if any audio on a Web page plays automatically for more than 3 seconds, either a mechanism is available to pause or stop the audio, or a mechanism is available to control audio volume independently from the overall system volume level (WCAG 1.4.2), time-based media is incorporated as the ability to change the viewer speed (WCAG 1.2), and pause, stop, hide functions are provided (WCAG 2.2.2).

FIGS. 4C and 4D illustrate an embodiment in which material is presented in a linear fashion with text-based function keys, including action prompts, according to an embodiment of the disclosure. As shown in FIGS. 4C and 4D, when the sequence in which content is presented affects its meaning, a correct reading sequence can be programmatically determined (WCAG 1.3.2); Instructions provided for understanding and operating content do not rely solely on sensory characteristics of components such as shape, color, size, visual location, orientation, or sound (WCAG 1.3.3); and color is not used as the only visual means of conveying information, indicating an action, prompting a response, or distinguishing a visual element (WCAG 1.4.1).

FIGS. 4E and 4F illustrate an embodiment in which operation can occur in landscape and portrait modes, according to an embodiment of the disclosure. As shown in FIGS. 4E and 4F, content does not restrict its view and operation to a single display orientation, such as portrait or landscape, unless a specific display orientation is essential. (WCAG 1.3.4).

FIGS. 4G and 4H illustrate an embodiment in which the purpose of input field is clearly defined, according to an embodiment of the disclosure. As shown in FIGS. 4G and 4H, the purpose of each input field collecting information about the user can be programmatically determined when: the input field serves a purpose identified in the input purposes for user interface components section, and the content is implemented using technologies with support for identifying the expected meaning for form input data. (WCAG 1.3.5).

FIGS. 4I-4L illustrate an embodiment in which the visual presentation of text and images of text has a contrast ratio of at least 4.5:1, according to an embodiment of the disclosure. As shown in FIGS. 4I-4L, the visual presentation of text and images of text has a contrast ratio of at least 4.5:1, except for the following: large text:large-scale text and images of large-scale text have a contrast ratio of at least 3:1. (WCAG 1.4.3).

FIGS. 4M and 4N illustrate an embodiment in which line height (line spacing) is at least 1.5 times the font size; spacing following paragraphs to at least 2 times the font size; letter spacing (tracking) to at least 0.12 times the font size; Word spacing to at least 0.16 times the font size, according to an embodiment of the disclosure. (WCAG 1.4.12).

FIG. 40 illustrates an embodiment in which hidden content incorporates a “dismiss” function rather than disappear with scrolling, according to an embodiment of the disclosure. As shown in FIG. 40, where receiving and then removing pointer hover or keyboard focus triggers additional content to become visible and then hidden, a mechanism is available to dismiss the additional content without moving pointer hover or keyboard focus. (WCAG 1.4.13).

FIGS. 4P and 4Q illustrate an embodiment in which titles, headings and labels describe topic or purpose, according to an embodiment of the disclosure. As shown in FIGS. 4P and 4Q, page titled web pages have titles that describe topic or purpose (WCAG 2.4.2) and headings and labels describe topic or purpose (WCAG 2.4.6).

FIGS. 4R and 4U illustrate an embodiment in which screen to screen sequence flow (rather than menus) are utilized for logic flow, according to an embodiment of the disclosure. As shown in FIGS. 4R and 4U, if a web page can be navigated sequentially and the navigation sequences affect meaning or operation, focusable components receive focus in an order that preserves meaning and operability (WCAG 2.4.3), and the purpose of each link can be determined from the link text alone or from the link text together with its programmatically determined link context, except where the purpose of the link would be ambiguous to users in general (WCAG 2.4.4).

FIGS. 4V and 4W illustrate an embodiment in which user interface components with labels that include text or images of text shall contain the text that is presented visually, according to an embodiment of the disclosure. (WCAG 2.5.3).

FIG. 4X illustrates an embodiment in which the human languages for phrases, passages or page can be selected by the user. (WCAG 3.1).

FIGS. 4Y-4BB illustrate an embodiment in which components that have the same functionality within a set of web pages are identified consistently and in similar order, according to an embodiment of the disclosure. As shown in FIGS. 4Y-4AA, navigational mechanisms that are repeated on multiple Web pages within a set of Web pages occur in the same relative order each time they are repeated, unless a change is initiated by the user, (WCAG 3.2.3) and components that have the same functionality within a set of web pages are identified consistently (WCAG 3.2.4).

FIGS. 4CC-4EE illustrate an embodiment in which labels or instructions shall be provided when content requires user input, according to an embodiment of the disclosure. (WCAG 3.3.2).

FIGS. 4FF-4HH illustrate an embodiment in which, when an input error is automatically detected, the item that is in error is identified and is described to the user in text (where applicable for security protection) along with corrective suggestions. As shown in 4FF-4HH, if an input error is automatically detected, the item that is in error is identified and the error is described to the user in text (WCAG 3.3.1), and if an input error is automatically detected and suggestions for correction are known, then the suggestions are provided to the user, unless it would jeopardize the security or purpose of the content (WCAG 3.3.3).

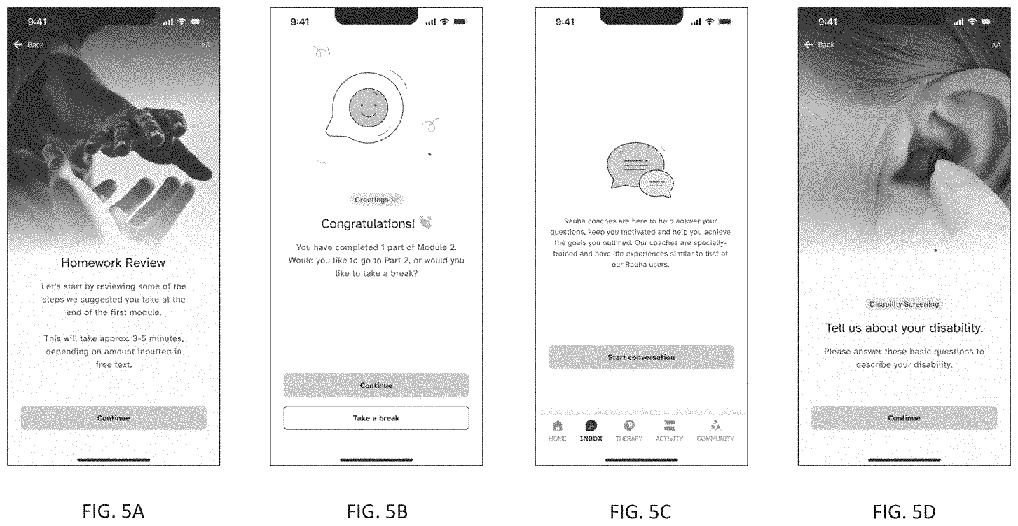

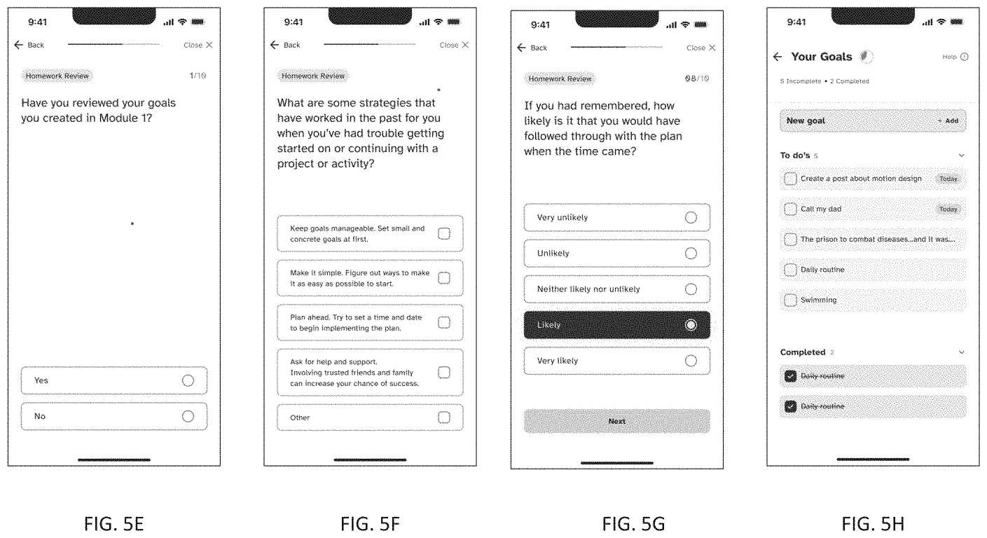

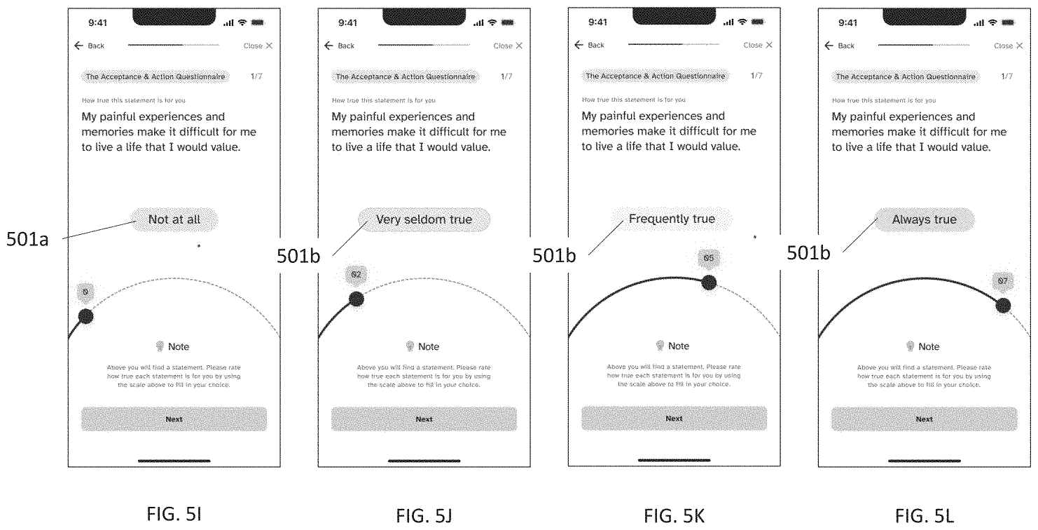

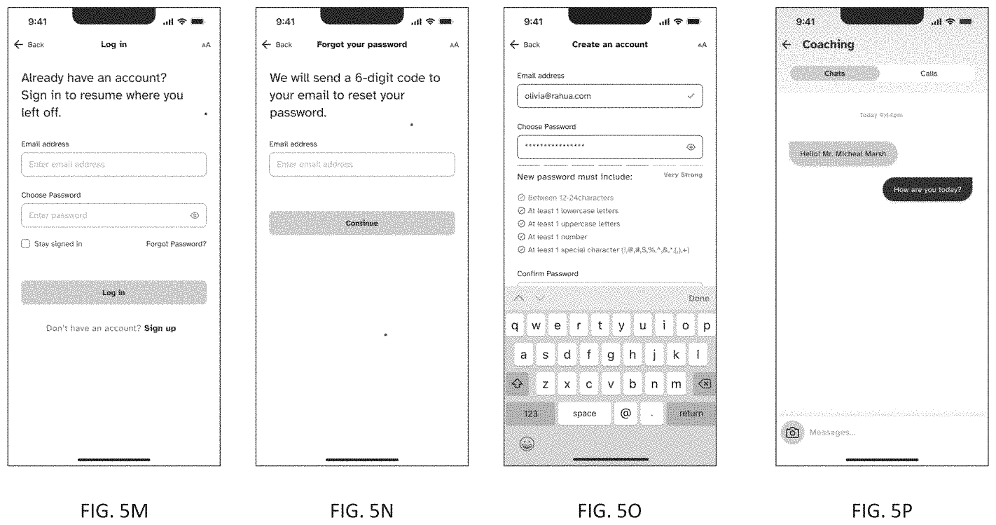

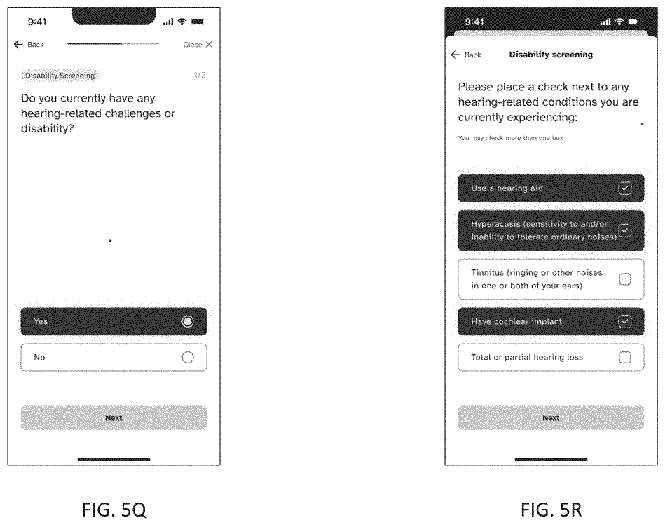

FIGS. 5A-5R show exemplary embodiments of screenshots of the platform's WCAG-compliant user interface, according to an embodiment of the disclosure. For example, FIGS. 5A-5D show predictive stop and start screens, according to an embodiment of the disclosure. FIGS. 5E-5H show click box entries. FIGS. 5I-5L show sliding scales with text/color changes, according to an embodiment of the disclosure. The text box referenced as 101a-b can be different colors depending on the content in the text box. For example, in some embodiments, the text box can be green when the content is “not at all” (FIG. 5I), gray when the content is “very seldom true” (FIG. 5J), yellow when the content is “frequently true” (FIG. 5K), and red when the content is “always true” (FIG. 5L). FIGS. 5M-5P show free text entries, according to an embodiment of the disclosure. FIGS. 5Q and 5R show self-identification for hearing, according to an embodiment of the disclosure.

COACHING—A well-documented problem in any medical intervention is the ability to stick with the treatment (retention). Patients start the therapy but then drop out. The dropout rate is particularly high in self-paced digital programs. It has been demonstrated that through the use of a personal coach, retention rates in digital therapy can be higher. In an embodiment, the platform includes a coaching platform and has a WCAG 2.1 designed communication platform so live coaches can interact with the patients. The coaches themselves are not (nor do they need to be) licensed therapists since therapy is driven by the CBT training modules.

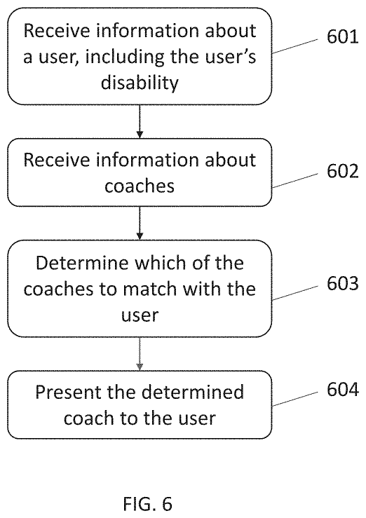

FIG. 6 is a flowchart of a method of matching a user with a coach, according to an embodiment of the disclosure. In some embodiments, the platform can perform a method of matching a user with a coach. A coach can be another user who can contact a user through the platform and encourage the user in the user's therapy journey. Having a coach may improve the user's engagement with the therapy provided by the platform.

A user and a plurality of coaches can each input his or her information into the platform. The information can include, for example, the user's disability, age, gender, sex, race, learning style, and socioeconomic information. At step 601, the method can include this receiving information. At step 602 and 603, the method can include determining which of the coaches should be matches with the user based off of the user's and the coaches' information. At step 604, the method can include presenting the determined coach to the user. The goal is to match a user with a coach who has the most in common with the user so that the coach understands the user can provide more targeted encouragement to the user. To make this determination, in some embodiments, the platform can use a machine learning algorithm to match a user and a coach. At step 604, the determined coach can be presented to the user.

In some embodiments, the platform can prompt one or more of the user and the plurality of coaches to complete disability surveys, and the platform may match a coach to a user (sometimes referred to as a “coachee”) based on the information in the disability survey. The disability survey can help paint a better picture of a user's or a coach's disability, impact and limitations. In some embodiments, the disability survey may ask for some or all of the following information: PHQ9, GAD 7, Work and Social Adjustment Scale (WSAS), which is designed to measure functional impairment attributable to an identified problem or disorder; AAQ-II, a 7-item measure of psychological inflexibility/experiential avoidance. Answers are given on a 7-point scale ranging from 1=never true to 7=always true; CCTS-10, Competencies of Cognitive Therapy Scale (CCTS) experiential avoidance; and WAI, the Working Alliance Inventory-Short Revised (WAI-SR) is a recently refined measure of the therapeutic alliance that assesses three key aspects of the therapeutic alliance: (a) agreement on the tasks of therapy, (b) agreement on the goals of therapy and (c) development of an affective bond.

The information can be leveraged with artificial intelligence to match compatible, chemistry and commonality. Taking the above information and matching coachees with a coach. The coaching can be integrated into learning paths along with elements like assessments, content, mentors and peer groups by leveraging its integration throughout the platform.

When matching a coach to a coachee, the following can be broadly taken into consideration: demographic information, some ICD9/10-based info, some personalization parameters. Additionally, questions regarding personal interests and hobbies can help to paint a more well-rounded picture of the user. In order to make good matches, it can be important to gather information on what participants hope to gain/give within the therapy. This can include questions such as: What skills would you like to improve upon within the program? What skills do you feel proficient in? What are your long-term goals? What values and qualities do you think are important for a peer supporters? For the peer supporters/coaches, what preferences are important to them in a match? It might be tenure or skillsets. Disability? Military or non military? Robust profiles (i.e., information about a user and a coach) can be needed to encompass a good range of information in order to make more informed matches. But keep in mind the profile shouldn't be too overwhelming for the participant. In some embodiments, questions are limited to no more than twelve questions per survey.

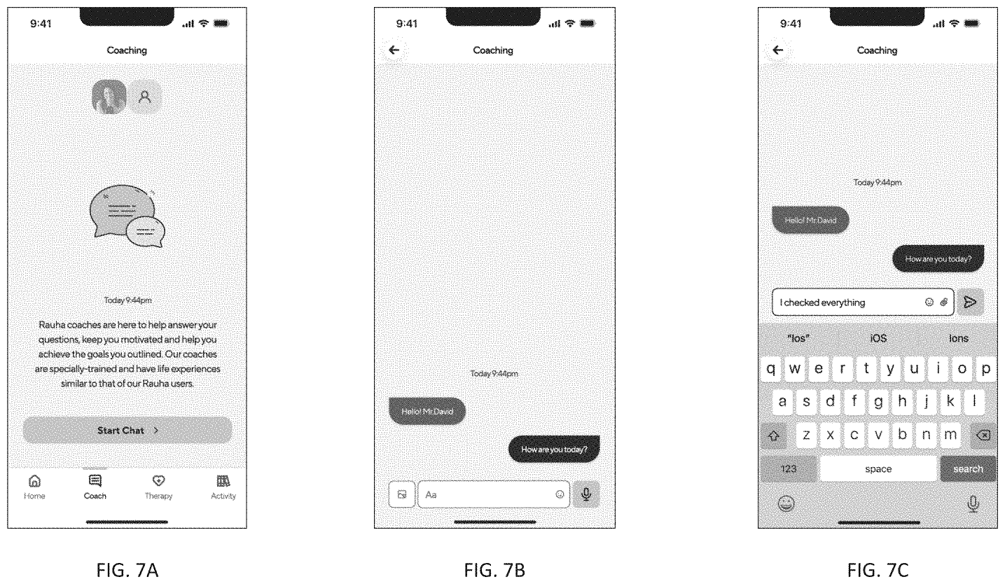

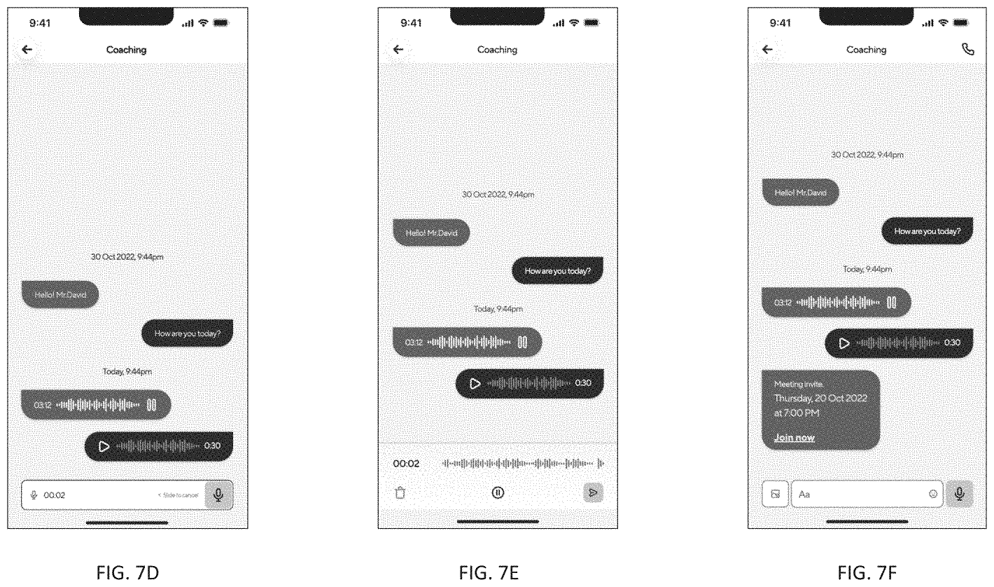

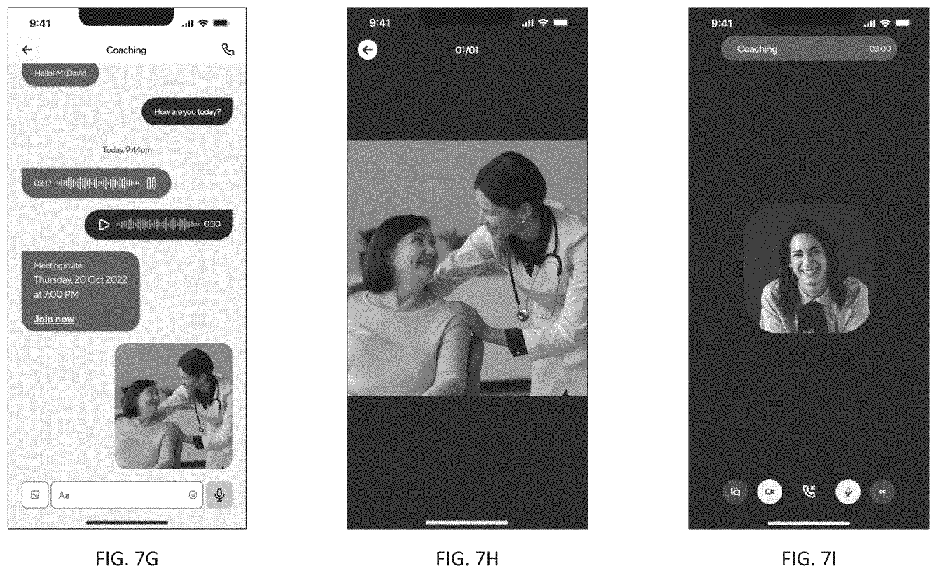

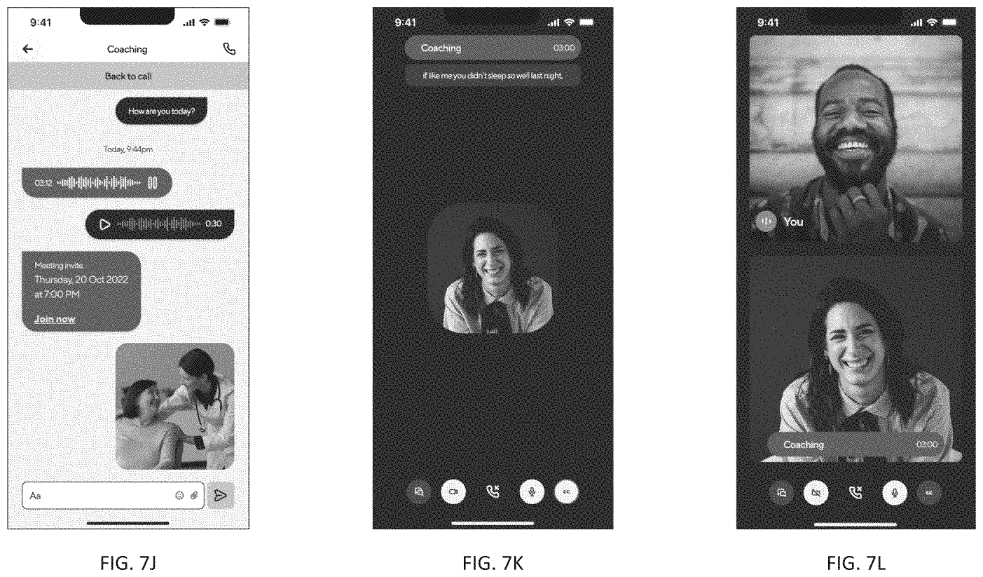

FIGS. 7A-7L illustrate how a user and a coach may communicate, according to an embodiment of the disclosure. As illustrated in FIGS. 7B and 7C, a coach and a user may send textual messages to one another. As illustrated in FIGS. 7D and 7E, a coach and a user may send audio messages to one another. As illustrated in 7F-7L, a coach and a user may schedule, join, and participate in video conferences with one another.

FORUMS—In some embodiments, the platform may include one or more community forums. The community forums can be disability-specific, meaning that each forum can be intended for discussing one or more particular disabilities. For example, a forum can be intended for users with a mobility impairment to discuss the therapy provided by the platform. In some embodiments, only those user's who have a particular disability can post in or view a forum intended for that particular disability. Having a forum where a user can connect with others going through the therapy may improve the user's engagement with the therapy provided by the platform.

MACHINE LEARNING—In some embodiments, the platform described herein utilizes machine learning algorithms. In an embodiment, the machine learning algorithm may be trained on the medical records for patients. The medical records may contain information related to a patient's type of disability, type of scenarios (ex., anxiety, depression, etc.), date and timestamps, activity type performing, geolocation, previous medical history, genetic history, and lifestyle. When a patient population uses the mobile application platform for the first time, based on their profile that includes a specific disability, a baseline may be created for that specific patient population. The machine learning algorithm may be trained in a two-step process. First, the machine learning algorithm may be trained on a patient's profile to create a “baseline.” Second, the machine learning algorithm may be trained on a set of data when the patient accesses the disability profile, type of scenarios, etc. With the integration of EMR, machine learning may also look out for any future hospital visits, procedures, lifestyle changes which will then be integrated with other data to provide a comprehensive assessment of intervention and scenario generation.

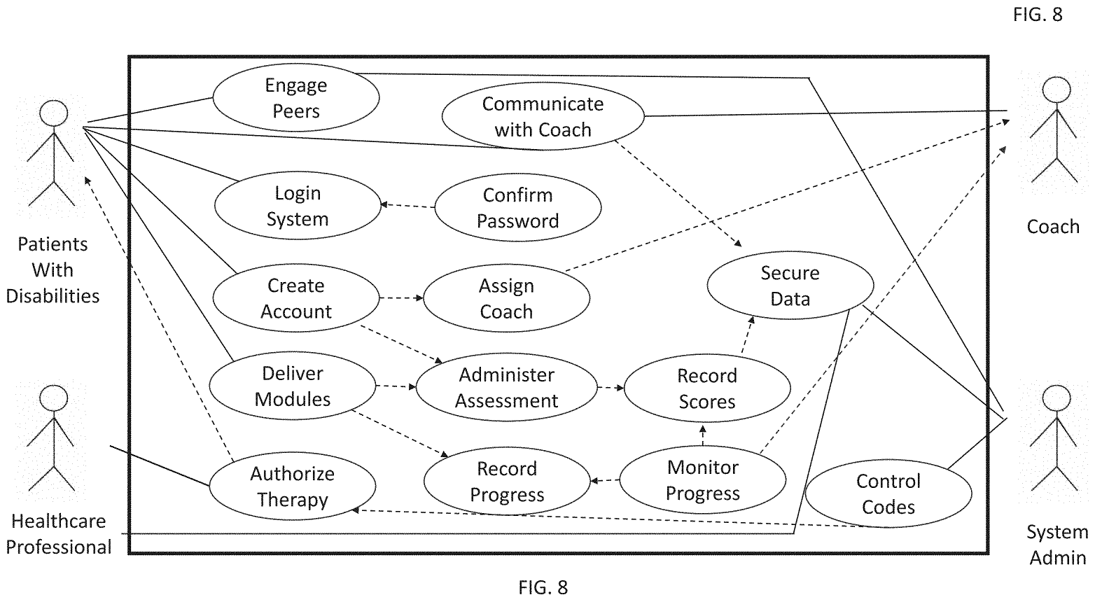

The next several figures and associated description describe the flow paths and architecture of the platform. FIG. 8 illustrates a use-case diagram of how the platform works, in an embodiment of the disclosure. By convention, the “customers” of the system are on the left, and the back-office operators are on the right. Solid lines are direct inputs while dotted lines represent the system responses. Things inside the box represent the system. As an example, consider authorization: a user can contact their health care provider outside of the platform. There is a direct line from the health care provider to “authorize therapy”: The health care provider authorizes a use code provided as part of marketing/sales outside the platform. There is a dotted line from “admin” to “authorize therapy”: The admin confirms a valid code. There is a straight line from “user” to “authorize therapy”: the user enters the code to open the system.

Once open, the user has straight line access to engaging peers, coaches, modules, and system login. Under “modules”, there is a dotted line to “administer assessments”: assessments are an automatic function of the modules. Under administer assessments, there are dotted lines to “record scores”: an auto feature of the assessments. Under “record scores,” there is a dotted line to “secure data”: an automatic feature of the scoring of assessments. Under “secure data” there is a solid line to the admin: The admin can see the scores. There is not a line to the coach from the scores; the coach cannot see the scores. There is a function to “monitor progress”: the coach can see how the cohort is progressing.

In some embodiments, the platform can have one or more of the following design features: easily accessed by the disability community, the capacity to “unlock” the program using a discrete key-code, the capacity for a secure sign in, data security for HIPPA Compliance, the ability of secure information to be shared as an EMR, can support CBT therapy in small, discrete doses, a capacity to disseminate media including Video, Audio and Infographics, a Capacity to record and track the evaluations, a capacity to perform Audio/Video Calls, the capacity to track and monitor completion of the modules with live coaching, the ability to start, stop and restart the various therapy modules, the Capacity for a User to have free-text journaling, a capacity to administer evaluations such as (GAD-7 or PHQ-9, understanding depression and anxiety experience), and the capacity to ensure suitability of this level of therapy among cohorts, the capacity to incorporate a badge/reward system.

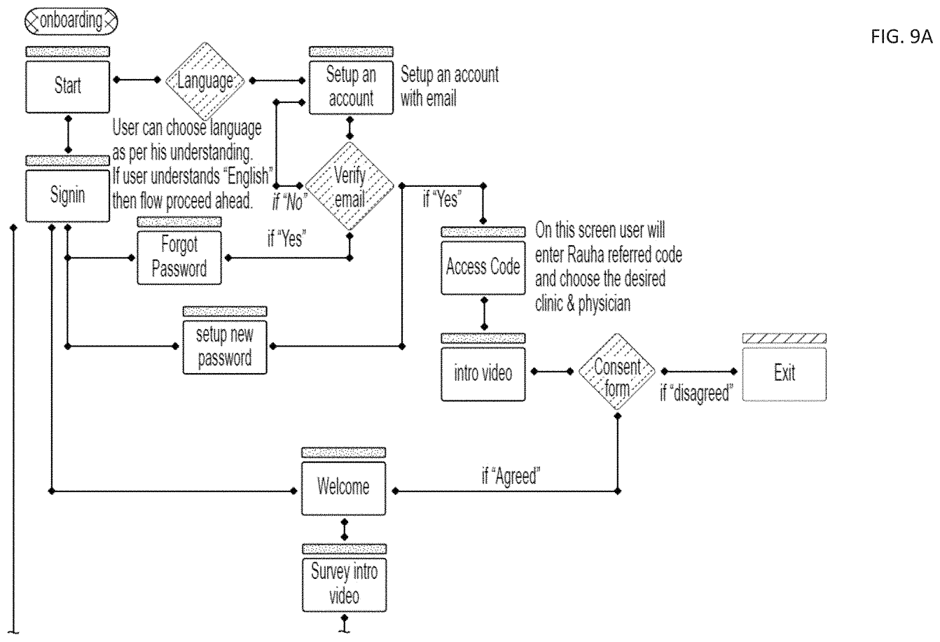

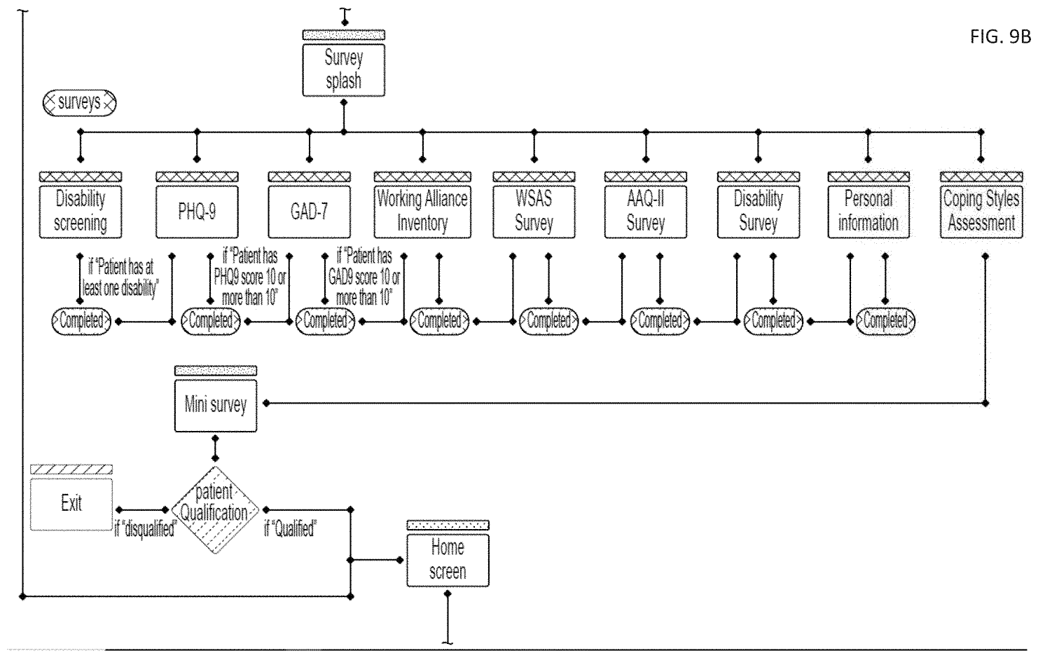

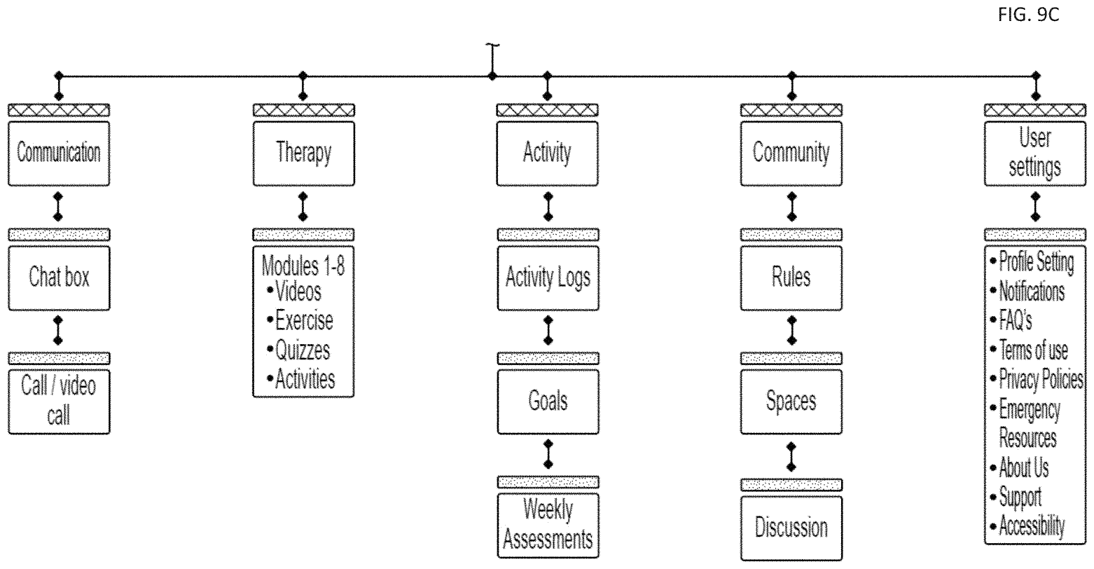

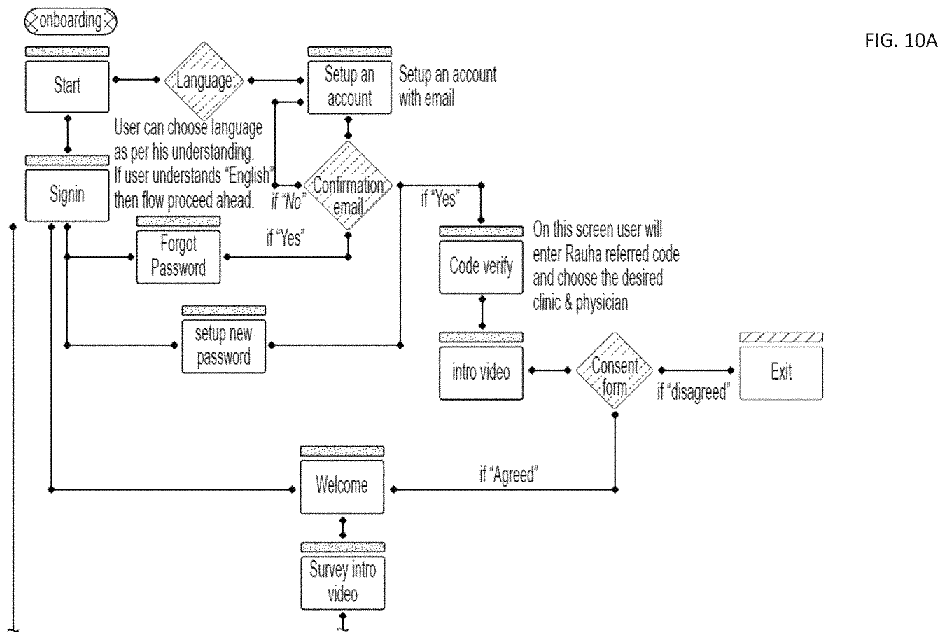

FIGS. 9A-9C illustrate the platform's master flow diagram, in accordance with an embodiment of the disclosure. Disability patient feedback may be incorporated on the application flow, features, instructional video clips, the personalization process, how to visualize performance over time, ideas for enhancing retention and preferences regarding the logistics of application use in daily life (e.g. how to schedule, reminder notices, etc.). What follows is a non-limiting example of how a patient may use the application: Patients will create an account. Receive a unique key-code from their HCP. Next, they will watch instructional videos, complete a personalization checklist, and complete their first weekly check-in. Coaches will have access to a dashboard. The dashboard will include the following features: weekly check-in, exercises, bonus mini-round, mood check-in, habit diary, and progress status. When patients are due for a check-in or an exercise, these buttons will text the patients and notify them on their user device.

Patients are prompted to complete a weekly self-assessment that includes re-personalization, a WSAP task with no corrective feedback, a mood check-in, and a habit diary. The mood check-in will include the Patient Health Questionnaire-9 (PHQ-9; Kroenke & Spitzer, 2002) and the General Anxiety Disorder-7 (GAD-7; Spitzer, Kroenke, Williams, & Lowe, 2006) so that users can self-monitor their symptoms. These measures for self-monitoring are brief, publicly available scales with excellent psychometric properties and are commonly used for routine clinical monitoring in real-world treatment settings. The habit diary prompts patients to think about their week and write free text about instances in which they catch themselves jumping to conclusions.

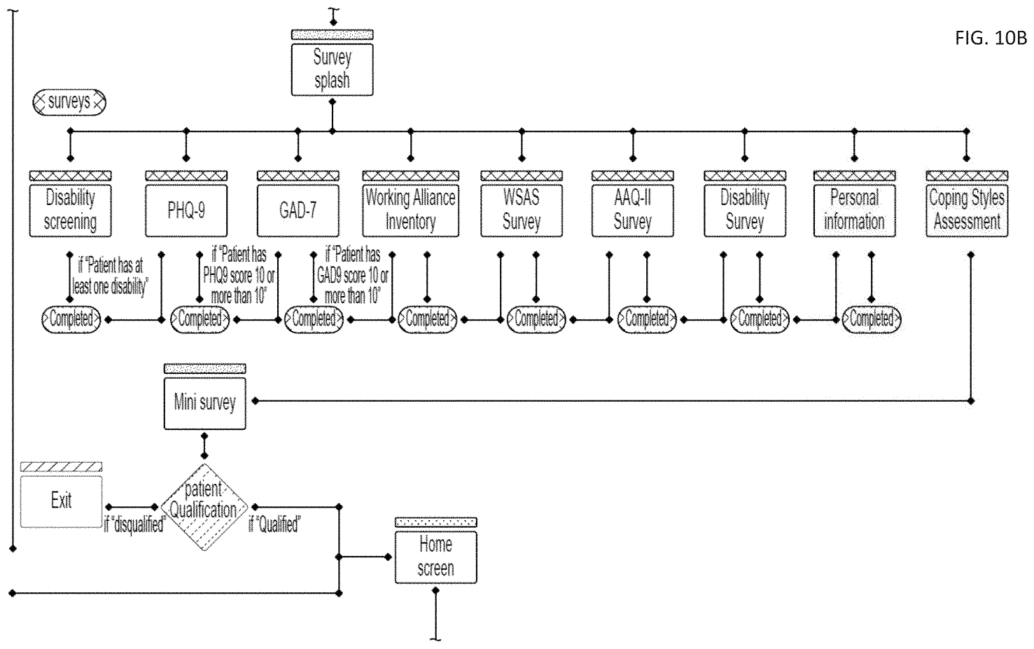

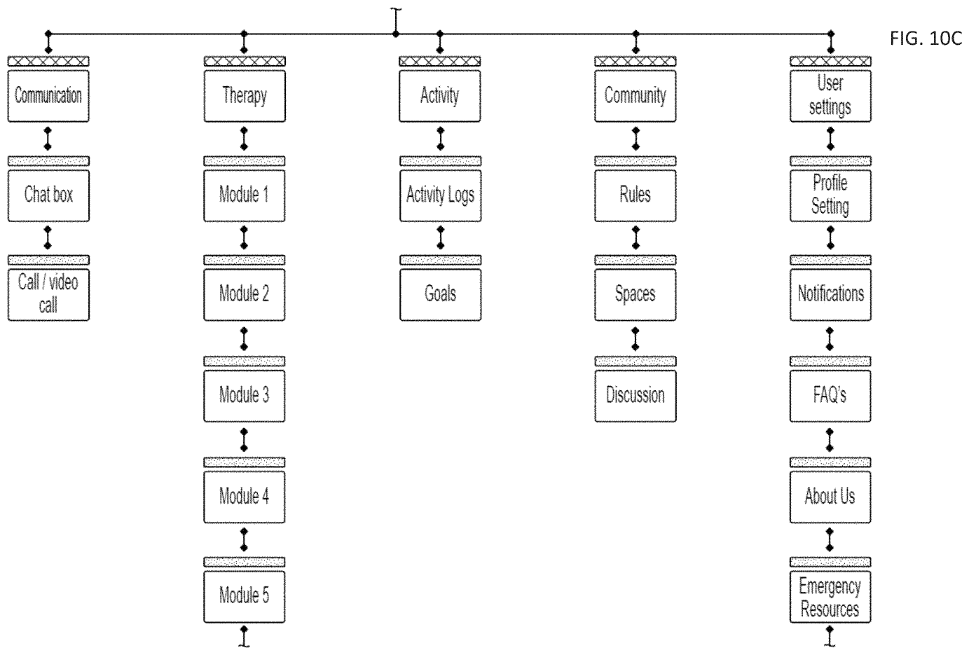

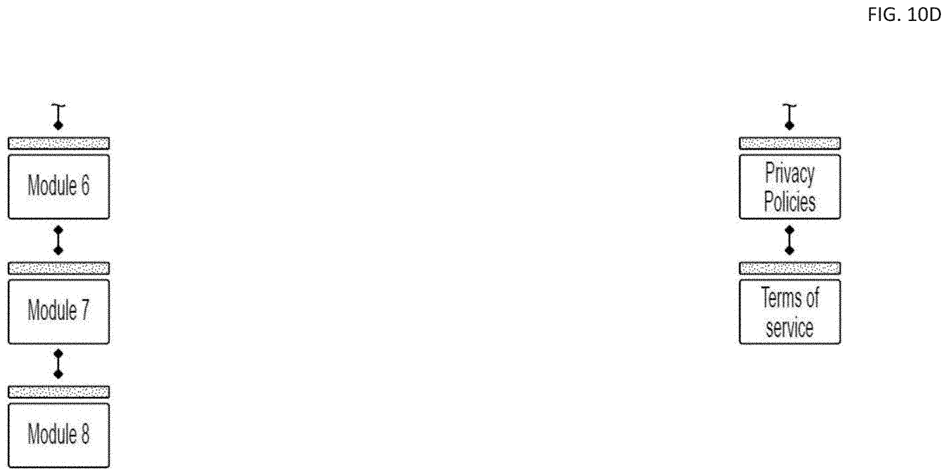

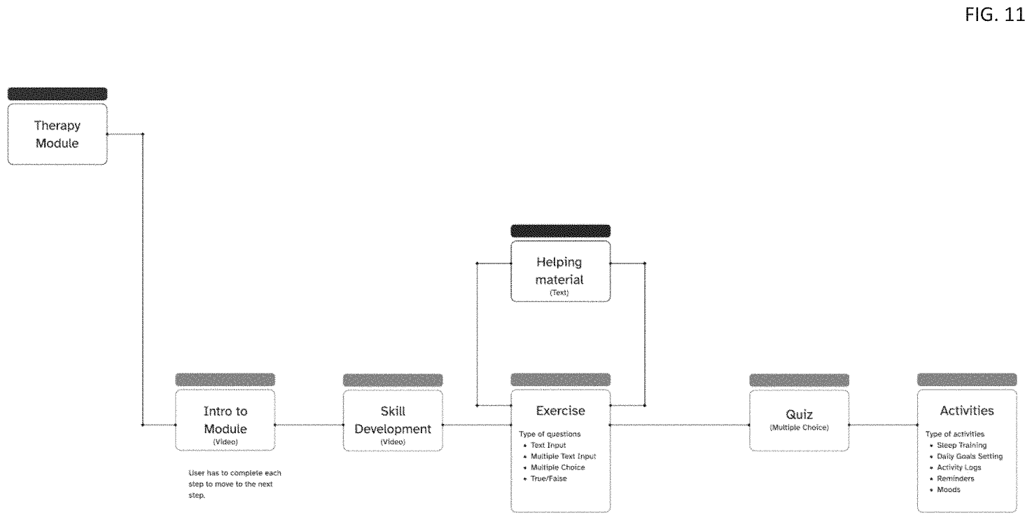

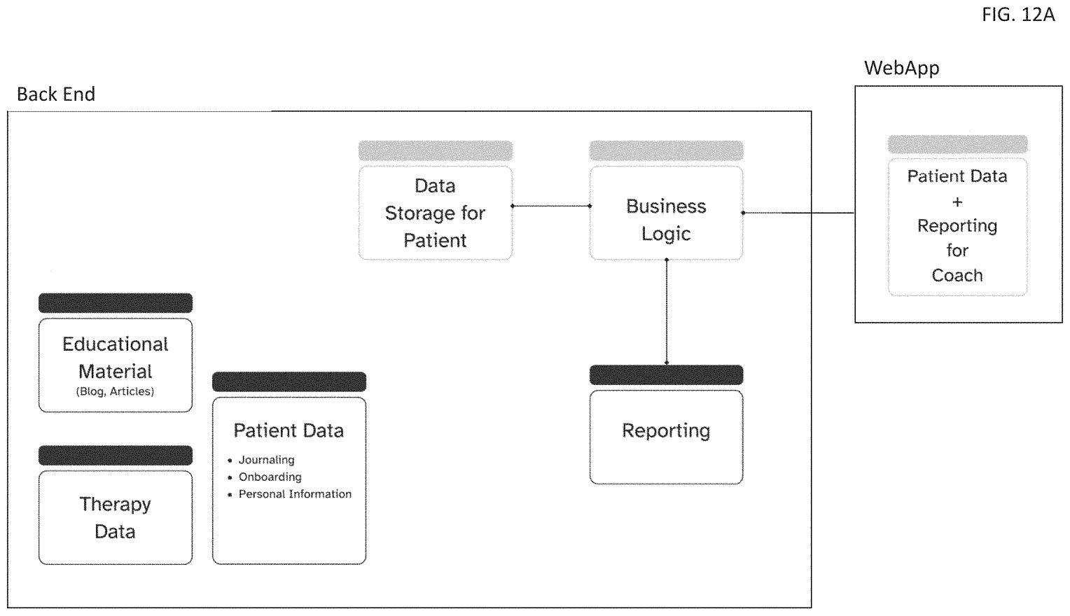

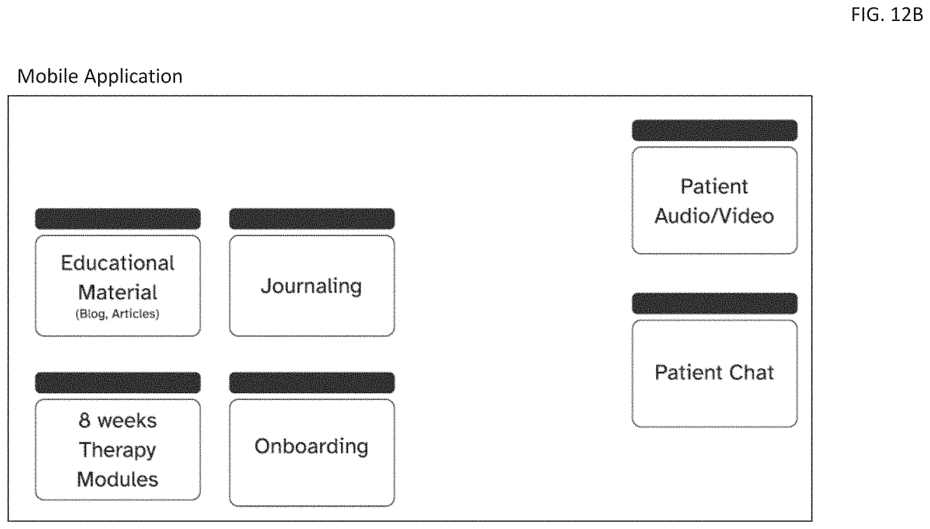

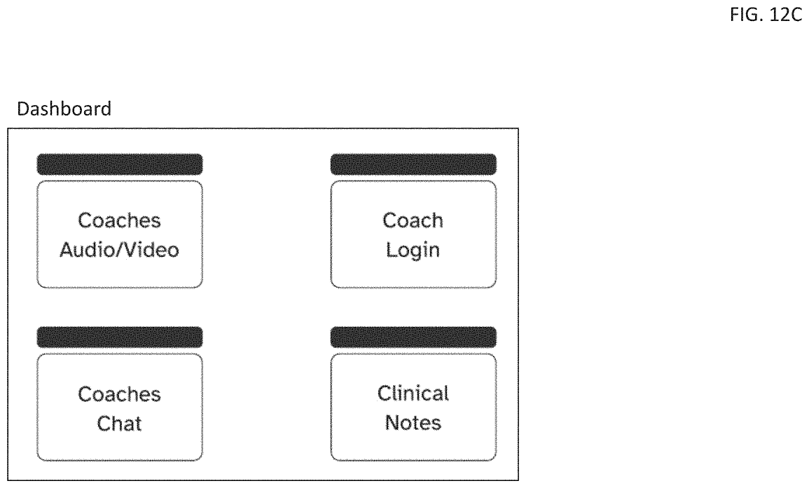

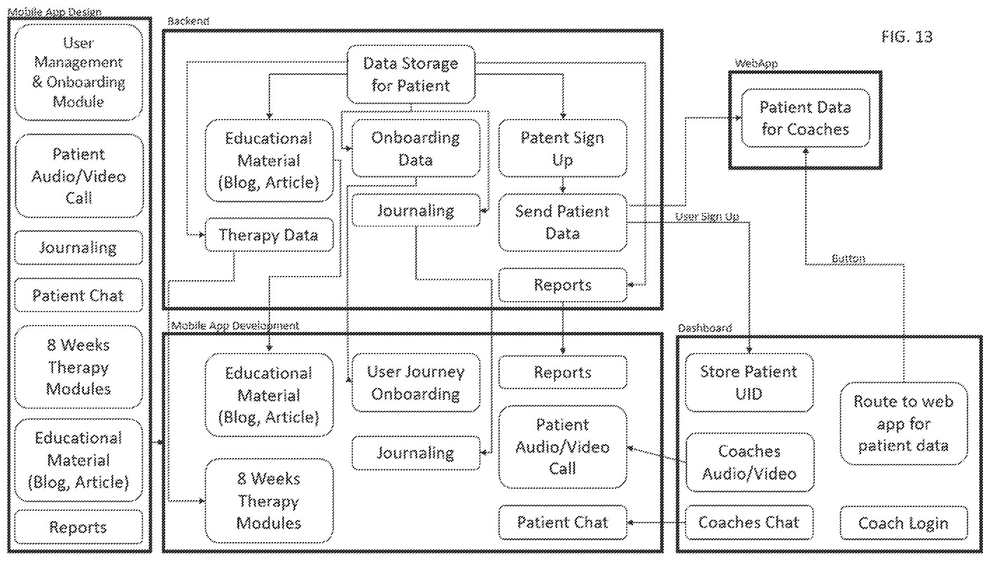

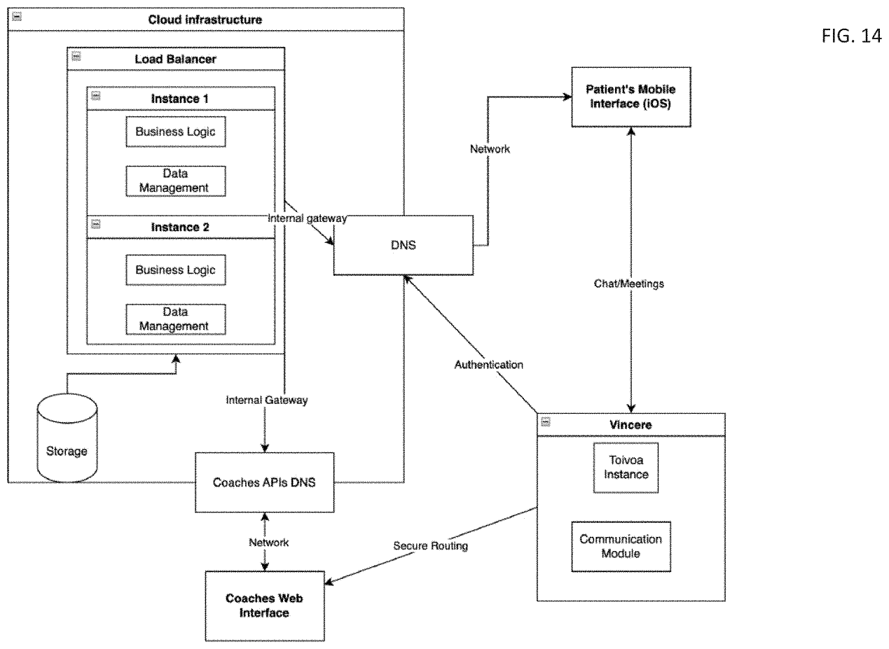

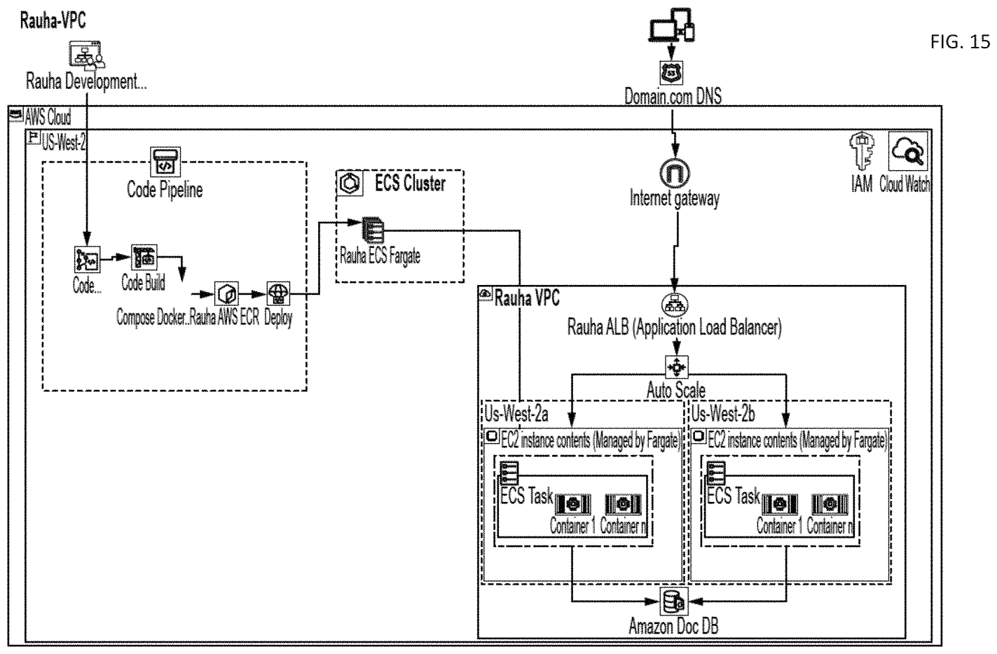

FIGS. 10A-10D illustrate the platform's information architecture (i.e., flow), according to an embodiment of the disclosure. FIG. 11 illustrates a sub-routine diagram, according to an embodiment of the disclosure. FIGS. 12A-12C illustrate the platform's architecture diagram, according to an embodiment of the disclosure. FIG. 12A illustrates the platform's backend and webapp architecture, according to an embodiment of the disclosure. FIG. 12B illustrates the platform's mobile application architecture, according to an embodiment of the disclosure. FIG. 12C illustrates the platform's dashboard architecture, according to an embodiment of the disclosure. FIG. 13 illustrates the platforms tech flow, according to an embodiment of the disclosure. FIG. 14 illustrates the platform's cloud infrastructure, according to an embodiment of the disclosure. FIG. 15 is the platform's VPC diagram, according to an embodiment of the disclosure.

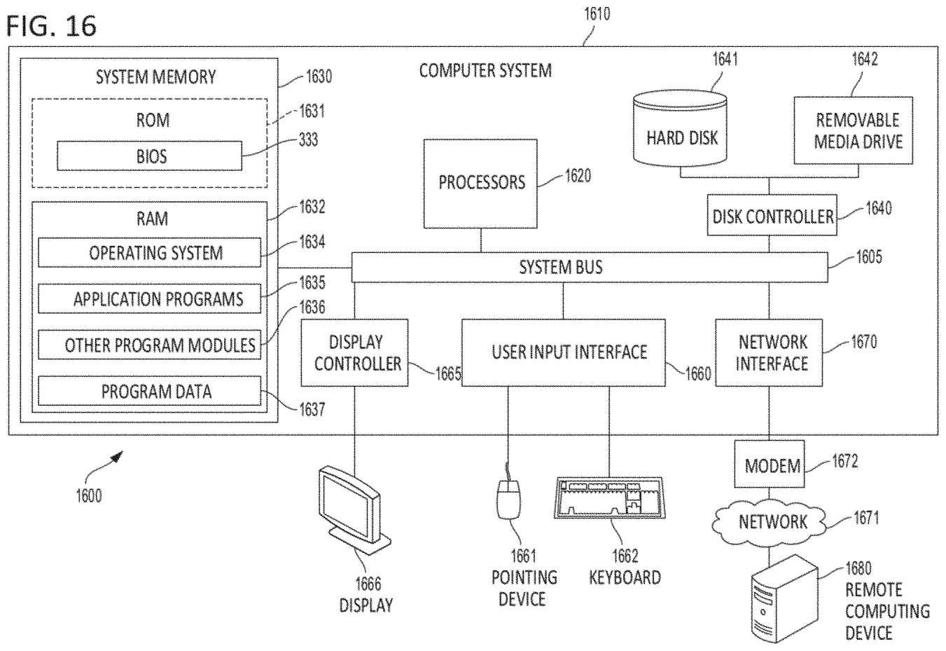

As shown in FIG. 16, the computer system 1510 may include a communication mechanism such as a bus 1521 or other communication mechanism for communicating information within the computer system 1510. The computer system 1510 further includes one or more processors 1520 coupled with the bus 1521 for processing the information. The processors 1520 may include one or more central processing units (CPUs), graphical processing units (GPUs), or any other processor known in the art.

The computer system 1610 also includes a system memory 1630 coupled to the bus 1621 for storing information and instructions to be executed by processors 1620. The system memory 1630 may include computer readable storage media in the form of volatile and/or nonvolatile memory, such as read only memory (ROM) 1631 and/or random access memory (RAM) 1632. The system memory RAM 1632 may include other dynamic storage device(s) (e.g., dynamic RAM, static RAM, and synchronous DRAM). The system memory ROM 1631 may include other static storage device(s) (e.g., programmable ROM, erasable PROM, and electrically erasable PROM). In addition, the system memory 1630 may be used for storing temporary variables or other intermediate information during the execution of instructions by the processors 1620. A basic input/output system (BIOS) 1633 containing the basic routines that help to transfer information between elements within computer system 1610, such as during start-up, may be stored in ROM 1631. RAM 1632 may contain data and/or program modules that are immediately accessible to and/or presently being operated on by the processors 1620. System memory 1630 may additionally include, for example, operating system 1634, application programs 1635, other program modules 1636 and program data 1637.

The computer system 1610 also includes a disk controller 1640 coupled to the bus 1621 to control one or more storage devices for storing information and instructions, such as a hard disk 1641 and a removable media drive 1642 (e.g., floppy disk drive, compact disc drive, tape drive, and/or solid state drive). The storage devices may be added to the computer system 1610 using an appropriate device interface (e.g., a small computer system interface (SCSI), integrated device electronics (IDE), Universal Serial Bus (USB), or FireWire).

The computer system 1610 may also include a display controller 1665 coupled to the bus 1621 to control a display 1666, such as a cathode ray tube (CRT) or liquid crystal display (LCD), for displaying information to a computer user. The computer system includes an input interface 1660 and one or more input devices, such as a keyboard 1662 and a pointing device 1661, for interacting with a computer user and providing information to the processor 1620. The pointing device 1661, for example, may be a mouse, a trackball, or a pointing stick for communicating direction information and command selections to the processor 1620 and for controlling cursor movement on the display 1666. The display 1666 may provide a touch screen interface which allows input to supplement or replace the communication of direction information and command selections by the pointing device 1661.

The computer system 1610 may perform a portion or all of the processing steps of embodiments of the invention in response to the processors 1620 executing one or more sequences of one or more instructions contained in a memory, such as the system memory 1630. Such instructions may be read into the system memory 1630 from another computer readable medium, such as a hard disk 1641 or a removable media drive 1642. The hard disk 1641 may contain one or more datastores and data files used by embodiments of the present invention. Datastore contents and data files may be encrypted to improve security. The processors 1620 may also be employed in a multi-processing arrangement to execute the one or more sequences of instructions contained in system memory 1630. In alternative embodiments, hard-wired circuitry may be used in place of or in combination with software instructions. Thus, embodiments are not limited to any specific combination of hardware circuitry and software.

As stated above, the computer system 1610 may include at least one computer readable medium or memory for holding instructions programmed according to embodiments of the invention and for containing data structures, tables, records, or other data described herein. The term “computer readable medium” as used herein refers to any medium that participates in providing instructions to the processor 1620 for execution. A computer readable medium may take many forms including, but not limited to, non-volatile media, volatile media, and transmission media. Non-limiting examples of non-volatile media include optical disks, solid state drives, magnetic disks, and magneto-optical disks, such as hard disk 1641 or removable media drive 1642. Non-limiting examples of volatile media include dynamic memory, such as system memory 1630. Non-limiting examples of transmission media include coaxial cables, copper wire, and fiber optics, including the wires that make up the bus 1621. Transmission media may also take the form of acoustic or light waves, such as those generated during radio wave and infrared data communications.

The computing environment 1600 may further include the computer system 1610 operating in a networked environment using logical connections to one or more remote computers, such as remote computer 1680. Remote computer 1680 may be a personal computer (laptop or desktop), a mobile device, a server, a router, a network PC, a peer device or other common network node, and typically includes many or all of the elements described above relative to computer system 1610. When used in a networking environment, computer system 1610 may include modem 1672 for establishing communications over a network 1671, such as the Internet. Modem 1672 may be connected to bus 1621 via user network interface 1670, or via another appropriate mechanism.

Network 1671 may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between computer system 1610 and other computers (e.g., remote computer 1680). The network 1671 may be wired, wireless or a combination thereof. Wired connections may be implemented using Ethernet, Universal Serial Bus (USB), RJ-11 or any other wired connection generally known in the art. Wireless connections may be implemented using Wi-Fi, WiMAX, and Bluetooth, infrared, cellular networks, satellite or any other wireless connection methodology generally known in the art. Additionally, several networks may work alone or in communication with each other to facilitate communication in the network 1671.

As one application of the exemplary computing environment 1600 to the technology described herein, consider an example system for analyzing DHM data which includes a network component, an image processing processor, and a GUI. The networking component may include network interface 1670 or some combination of hardware and software offering similar functionality. The networking component is configured to communicate with a DHM system to retrieve DHM images. Thus, in some embodiments, the networking component may include a specialized interface for communicating with DHM systems. The image processing processor is included in a computing system (e.g. computer system 1610) and is configured with instructions that enable to extract a reference image either from single object image or a time series of images received via the networking component, extract the regions from the object image where the fringe patterns are disturbed, and replace those regions with patterns existing from other parts of the image. The image processing processor may include additional functionality, as described in this disclosure, to support this task (e.g., segmentation, filling areas, etc.). The GUI may then be presented on a display (e.g., display 1666) for review by a user.

The embodiments of the present disclosure may be implemented with any combination of hardware and software. In addition, the embodiments of the present disclosure may be included in an article of manufacture (e.g., one or more computer program products) having, for example, computer-readable, non-transitory media. The media has embodied therein, for instance, computer readable program code for providing and facilitating the mechanisms of the embodiments of the present disclosure. The article of manufacture can be included as part of a computer system or sold separately.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

A graphical user interface (GUI), as used herein, comprises one or more display images, generated by a display processor and enabling user interaction with a processor or other device and associated data acquisition and processing functions. The GUI also includes an executable procedure or executable application. The executable procedure or executable application conditions the display processor to generate signals representing the GUI display images. These signals are supplied to a display device which displays the image for viewing by the user. The processor, under control of an executable procedure or executable application, manipulates the GUI display images in response to signals received from the input devices. In this way, the user may interact with the display image using the input devices, enabling user interaction with the processor or other device.

The functions and process steps herein may be performed automatically or wholly or partially in response to user command. An activity (including a step) performed automatically is performed in response to one or more executable instructions or device operation without user direct initiation of the activity.

The system and processes of the figures are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. As described herein, the various systems, subsystems, agents, managers and processes can be implemented using hardware components, software components, and/or combinations thereof. No claim element herein is to be construed under the provisions of 35 U.S.C. 112(f) unless the element is expressly recited using the phrase “means for.”

The system and processes of the figures are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. As described herein, the various systems, subsystems, agents, managers and processes can be implemented using hardware components, software components, and/or combinations thereof. No claim element herein is to be construed under the provisions of 35 U.S.C. 112(f) unless the element is expressly recited using the phrase “means for.”

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

Aspects of the present technical solutions are described herein with reference to flowchart illustrations and/or block diagrams of methods, apparatuses (systems), and computer program products according to embodiments of the technical solutions. It will be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present technical solutions. In this regard, each block in the flowchart or block diagrams can represent a module, segment, or portion of instructions, which includes one or more executable instructions for implementing the specified logical function(s). In some alternative implementations, the functions noted in the blocks can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

A second action can be said to be “in response to” a first action independent of whether the second action results directly or indirectly from the first action. The second action can occur at a substantially later time than the first action and still be in response to the first action. Similarly, the second action can be said to be in response to the first action even if intervening actions take place between the first action and the second action, and even if one or more of the intervening actions directly cause the second action to be performed. For example, a second action can be in response to a first action if the first action sets a flag and a third action later initiates the second action whenever the flag is set.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

We claim:

1. A system for providing disability-tailored therapy to a user having a disability with a presentation of the disability-tailored therapy compliant with accessibility requirements such that the presentation is accessible to the user having the disability, the system comprising:
a memory;
a processor coupled to the memory programed with executable instructions, the executable instructions cause the processor to obtain a disability-tailored therapy session for the user having the disability based on the disability; and
a user device configured to receive an input form the user and present the disability-tailored therapy session, wherein the input is user information comprising the disability, and wherein the presentation is configured to be accessible to the user having the disability.

2. The system of claim 1, wherein the presentation is further configured to be accessible to a second user having a second disability.

3. The system of claim 1, wherein the disability is a vision impairment, and wherein the instructions further cause the processor to adjust the presentation of the disability-tailored therapy session to input an audio output.

4. The system of claim 1,
wherein the executable instructions further cause the processor to match the user with one of a plurality of coaches, wherein each of the plurality of coaches is a person to encourage the user's engagement with the disability-tailored therapy, wherein matching the user with one of the plurality of coaches comprises:
receiving, by the processor, a plurality of coach information,
determining, by the processor, one of the plurality of coaches to match with the user using a machine learning algorithm,
presenting, by the user device, the determined coach

5. The system of claim 4,
wherein the user information further comprises the user's demographic information and learning style, and
wherein the plurality of coach information comprises each of the plurality of coaches' disability, demographic information and learning style.

6. The system of claim 1, wherein obtaining a disability-tailored therapy session for the user having the disability based on the disability comprises:
retrieving, by the processor from a database, a therapy session;
retrieving, by the processor from a database, a disability-specific input to insert into the therapy session; and
modifying, by the processor, the therapy session to include the disability-specific input;
wherein presenting, by the processor, the disability-tailored therapy session, comprises presenting the modified therapy session.

7. A computer-implemented method for providing disability-tailored therapy to a user having a disability with a presentation of the disability-tailored therapy that is compliant with accessibility requirements such that the presentation is accessible to the user having the disability in a data processing system comprising a processor and a memory comprising instructions that are executable by the processor, the method comprising:
receiving, by the processor, user information, wherein the user information comprises the disability of the user;
selecting, by the processor from a database, a disability-tailored therapy session based on the disability; and
presenting, by the processor, the disability-tailored therapy session, wherein the presentation is compliant with the accessibility requirements such that the disability-tailored therapy session is accessible to the user having the disability.

8. The computer-implemented method of claim 7, wherein the presentation is further configured to be accessible to a second user having a second disability.

9. The computer-implemented method of claim 7, wherein the therapy comprises cognitive behavioral therapy to treat one of anxiety and depression and the disability-tailored therapy session comprises a description of a situation intended to elicit an emotional response from the user.

10. The computer-implemented method of claim 7, wherein the disability is one of an impairment related to vision, hearing, and mobility.

11. The computer-implemented method of claim 7, wherein the disability is a vision impairment, the method further comprising:
Adjusting, by the processor, the presentation of the disability-tailored therapy session to input an audio output.

12. The computer-implemented method of claim 7, further comprising:
matching the user with one of a plurality of coaches, wherein each of the plurality of coaches is a person to encourage the user's engagement with the therapy, wherein matching the user comprises:
receiving, by the processor, a plurality of coach information, wherein each of the plurality of coach information is associated with one of a plurality of coaches,
determining, by the processor using a machine learning algorithm, one of the plurality of coaches to match with the user, and
presenting, by the processor to the user, the determined coach.

13. The computer-implemented method of claim 12,
wherein the user information further comprises the user's demographic information and learning style, and
wherein the plurality of coach information comprises each of the plurality of coaches' disability, demographic information and learning style.

14. The computer-implemented method of claim 7, wherein selecting a therapy session based on the disability further comprising:
retrieving, by the processor from a database, a therapy session;
retrieving, by the processor from a database, a disability-specific input to insert into the therapy session; and
modifying, by the processor, the therapy session to include the disability-specific input;
wherein presenting, by the processor, the disability-tailored therapy session, comprises presenting the modified therapy session.

15. A non-transitory computer-readable medium having stored thereon instructions for providing disability-tailored therapy to a user having a disability with a presentation of the disability-tailored therapy that is compliant with accessibility requirements such that the presentation is accessible to a person having the disability, which when executed by at least one processor, cause the processor to:
receive user information, wherein the user information comprises the disability of the user;
select, from a database, a first disability-tailored therapy session based on the disability; and
present the first disability-tailored therapy session, wherein the presentation is compliant with the accessibility requirements such that the first disability-tailored therapy session is accessible to the user having the disability.

16. The non-transitory computer-readable medium of claim 15, wherein the presentation is further configured to be accessible to a second user having a second disability.

17. The non-transitory computer-readable medium of claim 15, wherein the instructions further cause the processor to:
present a second disability-tailored therapy session,
wherein each of the first disability-tailored therapy session and the second disability-tailored therapy session have a respective purpose, and each respective purpose is to educate the user about one or more of a skill or a challenge in the user's treatment for one or more of anxiety and depression.

18. The non-transitory computer-readable medium of claim 15, wherein the instructions further cause the processor to:
match the user with one of a plurality of coaches, wherein each of the plurality of coaches is a person to encourage the user's engagement with the therapy, wherein matching the user comprises:
receiving a plurality of coach information, wherein each of the plurality of coach information is associated with one of a plurality of coaches,
determine, using a machine learning algorithm, one of the plurality of coaches to match with the user, and
present, to the user, the determined coach.

19. The non-transitory computer-readable medium of claim 18,
wherein the user information further comprises the user's demographic information and learning style, and
wherein the plurality of coach information comprises each of the plurality of coaches' disability, demographic information and learning style.

20. The non-transitory computer-readable medium of claim 15, wherein selecting the first disability-tailored therapy session based on the disability further comprises:
retrieving, by the processor from a database, an unmodified therapy session;
retrieving, by the processor from a database, a disability-specific input to insert into the unmodified therapy session; and
modifying, by the processor, the unmodified therapy session to include the disability-specific input;
wherein presenting, by the processor, the disability-tailored therapy session, comprises presenting the modified therapy session.
